(19) United States
(12) Patent Application Publication
(10) Pub. No.: US 2024/0246970 A1
(43) Pub. Date: Jul. 25, 2024

(54) SUBSTITUTED HETERO-BICYCLOCOMPOUNDS AS SUBTYPE SELECTIVE NICOTINIC ACETYLCHOLINE RECEPTOR INHIBITORS

(71) Applicant: THE BOARD OF TRUSTEES OF THE UNIVERSITY OF ILLINOIS, URBANA, IL (US)

(72) Inventors: ANDREW RILEY, CHICAGO, IL (US); MALAIKA ARGADE, CHICAGO, IL (US); LISA RUSALI, CHICAGO, IL (US); CAROLYN STRAUB, CHICAGO, IL (US); KYLE KREMILLER, CHICAGO, IL (US)

(21) Appl. No.: 18/580,788

(22) PCT Filed: Jul. 21, 2022
(86) PCT No.: PCT/US2022/073998

§ 371 Date: Jan. 19, 2024

Related U.S. Application Data
(60) Provisional application No. 63/321,145, filed on Mar. 18, 2022.
(60) Provisional application No. 63/224,115, filed on Jul. 21, 2021.

(51) Int. Cl. C07D 471/04; A61K 31/439; A61K 31/444; A61K 31/4709; A61K 31/4725; C07D 225/06; C07D 401/04; C07D 405/04

(52) U.S. Cl. CPC C07D 471/04; A61K 31/439; A61K 31/444; A61K 31/4709; A61K 31/4725; C07D 225/06; C07D 401/04; C07D 405/04

(57) ABSTRACT

In one aspect, the disclosure relates to a scaffold for a class of small molecules that selectively inhibit the α3β4 nicotinic acetylcholine receptor (nAChR) subtype, as well as molecules constructed using the scaffold and syntheses thereof. In some aspects, the scaffold can be used as a basis for synthesizing additional molecules capable of selectively inhibiting other nAChR subtypes. In a further aspect, the disclosed small molecules can be used as molecular probes to investigate the function of different nAChR subtypes and as potential treatments for addiction and other diseases.

### CROSS REFERENCE TO RELATED APPLICATIONS

This application claims the benefit of U.S. Provisional Application No. 63/224,115 filed on Jul. 21, 2021, and U.S. Provisional Application No. 63/321,145 filed on Mar. 18, 2022, each of which is incorporated herein by reference in its entirety.

### STATEMENT REGARDING FEDERALLY SPONSORED RESEARCH OR DEVELOPMENT

This invention was made with government support under grant number Al116441 awarded by the National Institutes of Health. The government has certain rights in this invention.

### BACKGROUND

Plants of the Aristotelia genus produce a family of alkaloids that feature indoles bonded to a monoterpene. In most monoterpene indole alkaloids, the terpenoid portion is derived from secologanin. In contrast, the Aristotelia alkaloids incorporate tryptamine with a non-rearranged geranyl unit. The resulting Aristotelia indole alkaloids possess a characteristic 3-azabicyclo[3.3.1]nonane architecture, as in hobartine (FIG. 1). Additional family members such as peduncularine and aristoteline arise through cyclization with and rearrangements of the azabicyclic core. The related quinoline alkaloid aristoquinoline (1) was isolated from the leaves of Aristotelia chilensis in 1993. The biological activity of 1 went uninvestigated for >25 years, but recently, Arias et al. determined that 1 is an antagonist of the nicotinic acetylcholine receptors (nAChRs). Notably, 1 demonstrated an uncommon and desirable preference for the α3β4 subtype of nAChRs, which have been proposed as targets to treat a variety of substance use disorders.

Driven by this rare subtype selectivity, an efficient synthesis of 1 was developed to characterize its biological activity. However, unlike the indole-containing Aristotelia alkaloids, whose absolute configurations have been confirmed by X-ray crystallography and total synthesis, neither the absolute configuration nor the specific rotation of 1 has been reported. Moreover, the configuration of the C9 stereogenic center reported by Arias differs from that originally proposed by Cespedes. Thus, while the configuration of 1 depicted in FIG. 1 may be inferred from other Aristotelia alkaloids, the relative and absolute configurations of 1 have never before been determined.

The nicotinic acetylcholine receptors (nAChRs) are a family of ionic channels found throughout the central and peripheral nervous system, where they play an important role in responding to the neurotransmitter acetylcholine. In humans there are 8 alpha and 3 beta subunits that assemble into different combinations of alpha and/or beta subunits; these combinations are known as nAChRs subtypes. Different nAChRs subtypes are found in different regions of the nervous system and thus play different roles in normal function and different diseases. For instance, the a3b4 subtype found in regions in the brain associated with drug addiction. Although compounds that inhibit the receptors are known-some of which have been used clinically-few are able to selectively inhibit specific subtypes. There is currently a lack of small molecules that inhibit different nAChR subtypes with a high degree of selectivity. Those that do exist generally suffer from poor drug-like properties that prevent their use in the clinic. Thus, it would be desirable to develop small molecules having improved drug-like properties for use in conditions associated with aberrant nAChR activity.

Despite advances in research relating to small molecule inhibitors of nAChR, there is still a scarcity of compounds that are potent, efficacious, and selective inhibitors of specific nAChR subtypes, and also effective in the treatment of diseases and disorders associated with aberrant nAChR activity. These needs and other needs are satisfied by the present disclosure.

### SUMMARY

In accordance with the purpose(s) of the present disclosure, as embodied and broadly described herein, the disclosure, in one aspect, relates to a scaffold for a class of small molecules that selectively inhibit the α3β4 nicotinic acetylcholine receptor (nAChR) subtype, as well as molecules constructed using the scaffold and syntheses thereof. In some aspects, the scaffold can be used as a basis for synthesizing additional molecules capable of selectively inhibiting other nAChR subtypes. In a further aspect, the disclosed small molecules can be used as molecular probes to investigate the function of different nAChR subtypes and as potential treatments for addiction and other diseases.

In one aspect, the disclosed compounds are compounds of Formula I or a pharmaceutically acceptable salt thereof:

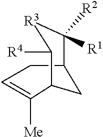

wherein R¹ and R² are independently selected from hydrogen, C1-C4 alkyl, C1-C4 haloalkyl, C3-C6 cycloalkyl, phenyl, and deuterated C1-C4 alkyl, or wherein R¹ and R² together are part of a cycloalkyl group;
wherein R³ is selected from NR⁵, S, and O;
   wherein R⁵ is selected from hydrogen, C1-C4 alkyl, and acetyl; and
wherein R⁴ is selected from (1) a C1-C4 alkyl or alkenyl group substituted with a substituted or unsubstituted aryl group or (2) a substituted or unsubstituted 5 to 10-membered aryl or heteroaryl group;
provided that when R¹ and R² are methyl and R³ is NH, R⁴ is not

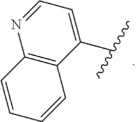 [SMILES: *c1ccnc2ccccc12]

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows convergent Ritter-like reactions between monoterpenes and nitriles are employed to synthesize the characteristic Aristotelia azabicyclic scaffold.

FIGS. 2A-2B show dual agonist/antagonist fluorescence assays. For calcium measurements, fluorescence measurements (Ex: 485 nM, Em: 525, Cutoff: 515 nM) were taken for 20 seconds prior to addition of compounds (50 μL of HBSS+0.1% DMSO solution at 5× final concentration) using a FlexStation 3 followed by 90 seconds of continuous measurements. For agonist assays, 12 concentrations of test compounds were added to the cells (300 μM-100 μM, final concentrations). Epibatidine (30 μM-10 μM) and mecamylamine (150 μM-50 μM) were used as agonists (FIG. 2A) and antagonist (FIG. 2B) positive controls.

FIGS. 3A-3B show concentration response curves. Ligand response for both agonist (FIG. 3A) and antagonist (FIG. 3B) data was calculated by subtracting the average of the pre-addition fluorescence (Fₒ) from the maximum response (Fₘₐₓ) for each well, correcting to a DMSO control, and normalizing to the maximal response produced by epibatidine. Each concentration-response point represents a minimum of two technical replicates and three biological replicates. EC₅₀, Eₘₐₓ, and IC₅₀ values were calculated from corresponding concentration-response curves fitted using the logistical curve in OriginPro.

FIG. 4 shows an X-ray crystal structure of aristoquinoline ((−)−1).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are compounds of Formula I or a pharmaceutically acceptable salt thereof

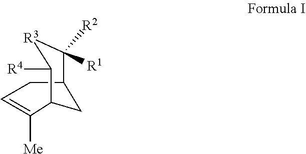

wherein R¹ and R² are independently selected from hydrogen, C1-C4 alkyl, C1-C4 haloalkyl, C3-C6 cycloalkyl, phenyl, and deuterated C1-C4 alkyl, or wherein R¹ and R² together are part of a cycloalkyl group;
wherein R³ is selected from NR⁵, S, and O;
   wherein R⁵ is selected from hydrogen, C1-C4 alkyl, and acetyl; and
wherein R⁴ is selected from (1) a C1-C4 alkyl or alkenyl group substituted with a substituted or unsubstituted aryl group or (2) a substituted or unsubstituted 5 to 10-membered aryl or heteroaryl group;
provided that when R¹ and R² are methyl and R³ is NH, R⁴ is not

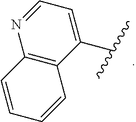 [SMILES: *c1ccnc2ccccc12]

In another aspect, disclosed herein is the compound of Formula I, wherein R¹ and R² are independently selected from hydrogen, methyl, and ethyl. In some aspects, R¹ and R² are both methyl, or are together both part of a cyclopropyl group.

In still another aspect, R³ can be NR⁵ and R⁵ can be selected from hydrogen, methyl, or ethyl. In another aspect, R³ can be O. In some aspects, in the compounds of Formula I, R¹ and R² are methyl and R³ is NH.

In still another aspect, R⁴ is a phenyl group substituted with one or more of: hydroxyl, nitro, amino, halo, C₁-C₄ haloalkyl, C₁-C₄ alkoxy, or C₁-C₄ alkyl, C3-C6 cycloalkyl, or any combination thereof, or a quinoline group substituted with one or more of: hydroxyl, nitro, amino, halo, C₁-C₄ haloalkyl, C₁-C₄ alkoxy, or C₁-C₄ alkyl, C3-C6 cycloalkyl, or any combination thereof.

In another aspect, R⁴ can be

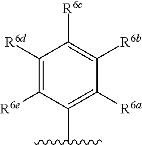

and R⁶, R^{6b}, R^{6c}, R^{6d}, and R^{6e} are independently selected from hydrogen, halogen, hydroxyl, N-acetyl, —OCF₃, nitro, isopropyl, cyclopropyl, tert-butyl, dimethylamino, or any combination thereof.

In still another aspect, R⁴ can be

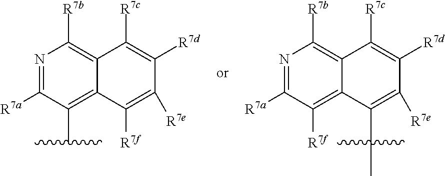

and R^{7a}, R^{7b}, R^{7c}, R^{7d}, R^{7e}, and R^{7f} are independently selected from hydrogen, halogen, hydroxyl, N-acetyl, —OCF₃, nitro, isopropyl, cyclopropyl, tert-butyl, dimethylamino, or any combination thereof.

In any of these aspects, R⁴ can be selected from

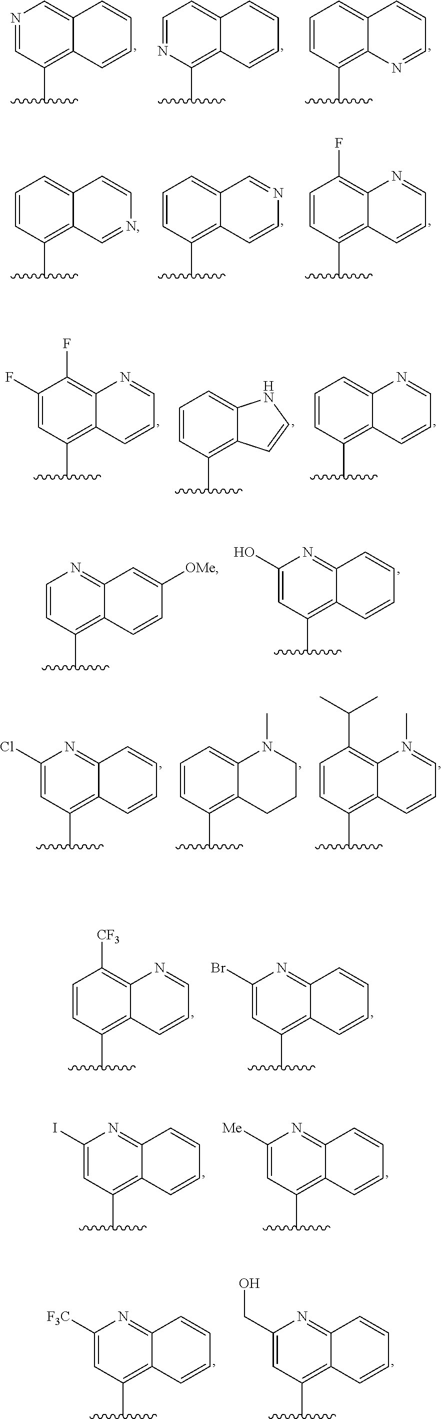

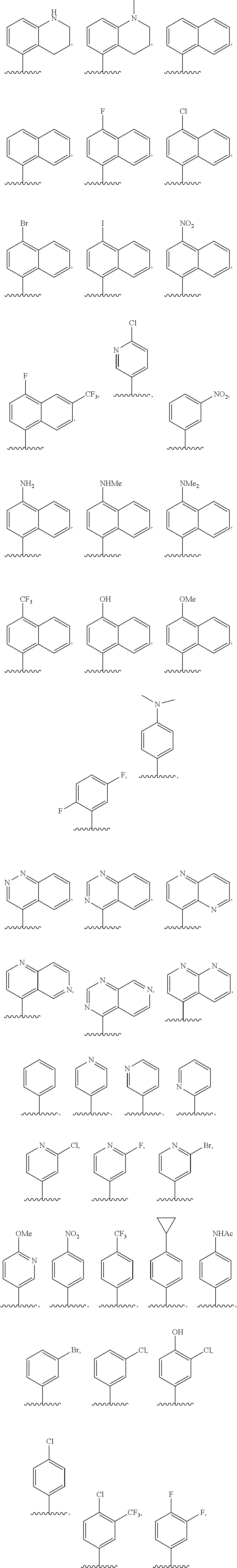

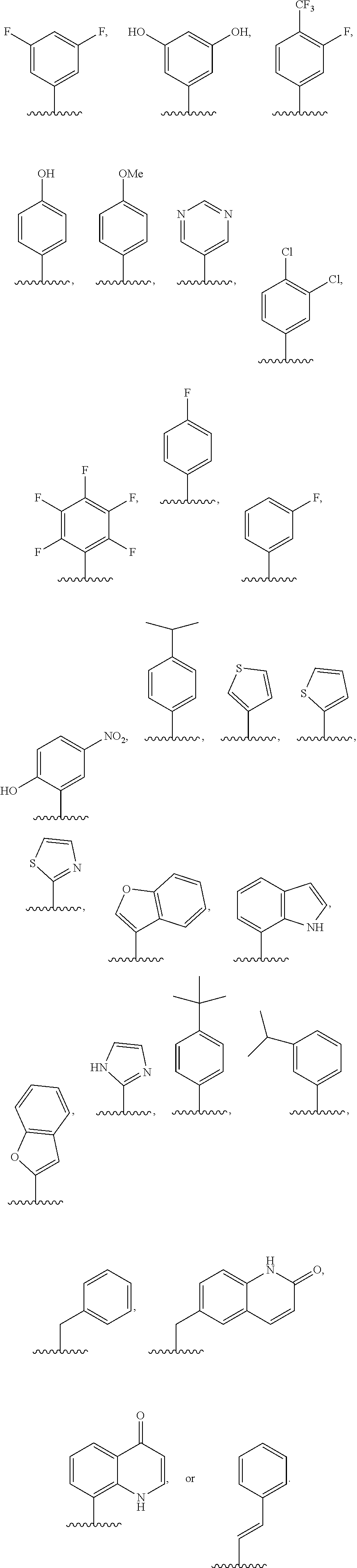

In one aspect, the compound of Formula I is

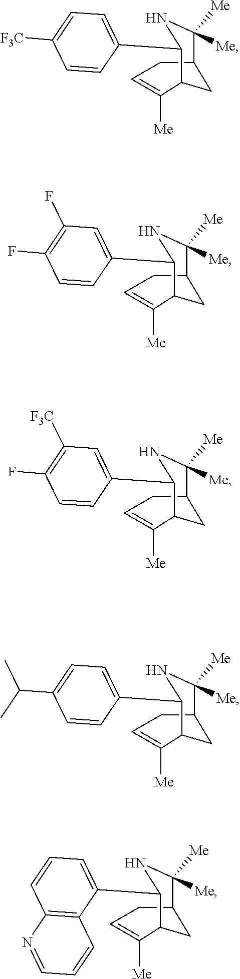

or a pharmaceutically acceptable salt thereof, or is

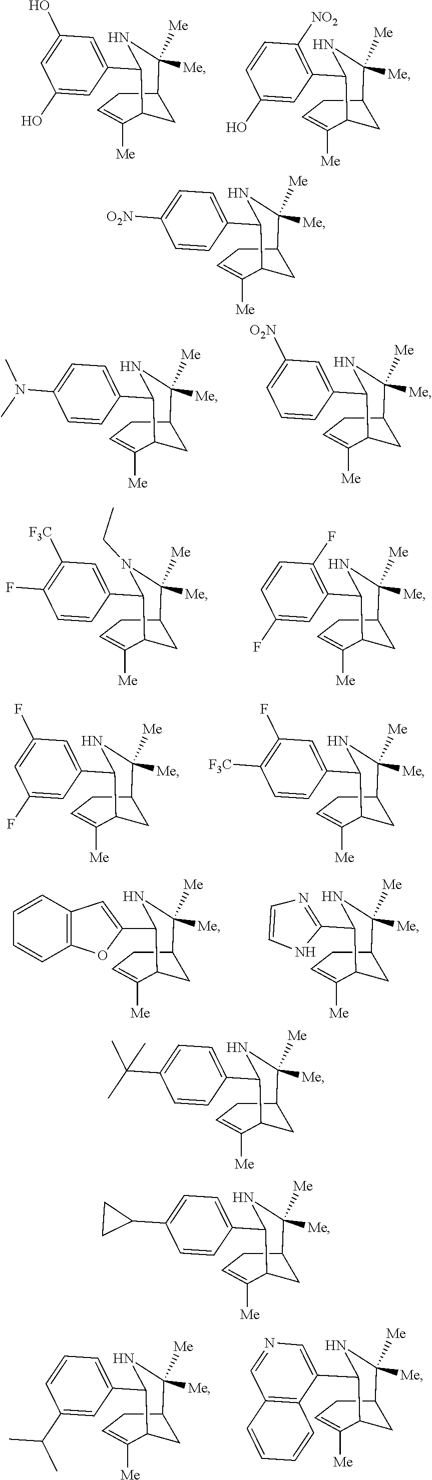

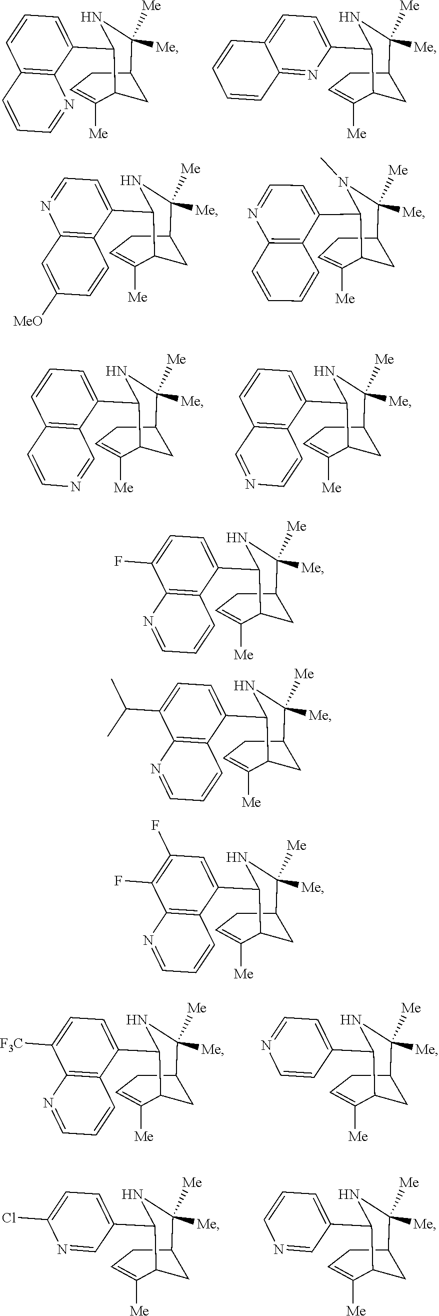

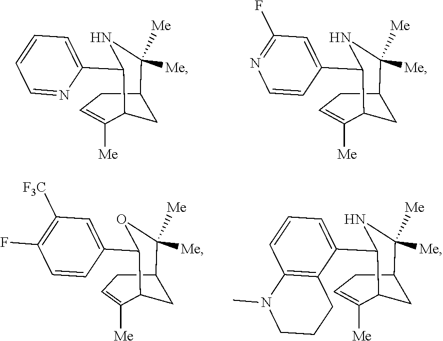

or a pharmaceutically acceptable salt thereof. In yet another aspect, the compound of Formula I is

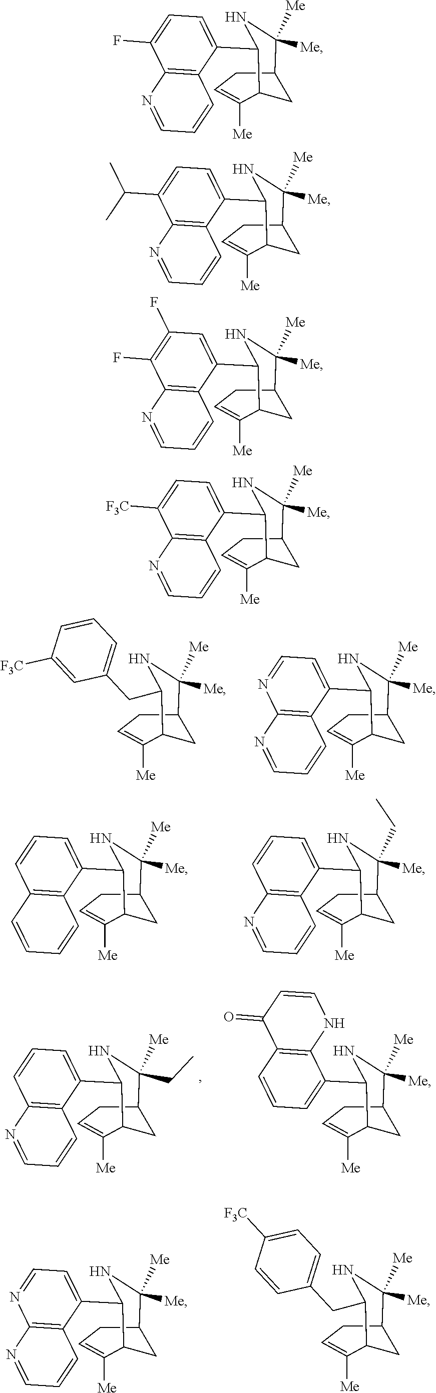

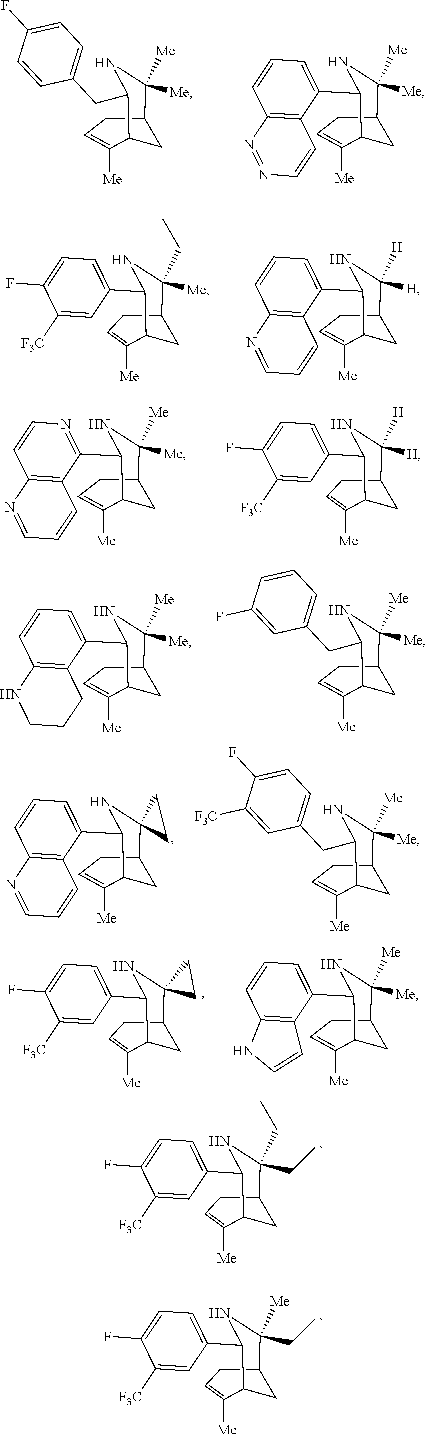

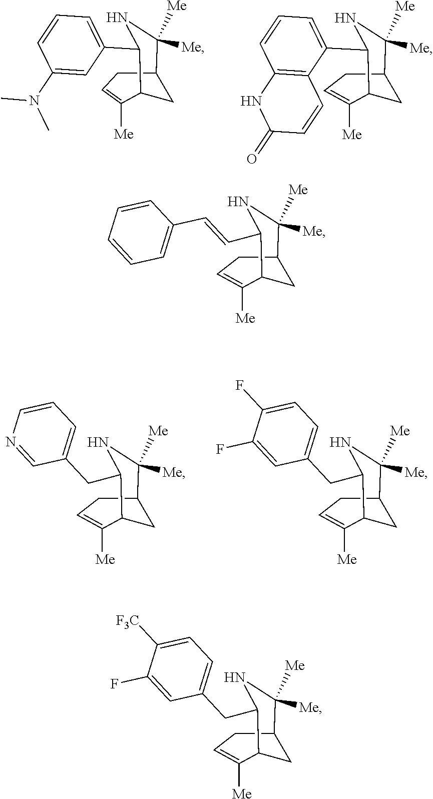

or a pharmaceutically acceptable salt thereof.

In an aspect, the compound can be a (+) enantiomer, a (−) enantiomer, or any combination thereof, where (+) and (−) enantiomers are determined by measuring optical rotation of the compound using a polarimeter. In one aspect, the compound of Formula I has a chiral center a

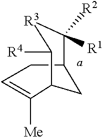

and the stereochemistry at chiral center a is substantially R, substantially S, or racemic.

Also disclosed herein is a method for inhibiting the activity of at least one nicotinic acetylcholine receptor (nAChR) subtype, the method including contacting the nAChR subtype with a disclosed compound. In a further aspect, the at least one nAChR subtype is an α3β4 nAChR. In still another aspect, the compound has an IC₅₀ for the nAChR subtype of less than about 1 μM. In a further aspect, the compound has an IC₅₀ for the nAChR subtype of from about 0.25 to about 0.5 μM.

Furthermore, disclosed herein are pharmaceutical compositions including a disclosed compound, an enantiomer thereof, or a pharmaceutically acceptable salt thereof, and a pharmaceutically acceptable carrier or excipient.

In one aspect, disclosed herein is method for treating or preventing a disease or disorder associated with aberrant activity of at least one nicotinic acetylcholine receptor (nAChR) subtype in a subject, the method including administering a therapeutically effective amount a disclosed compound or pharmaceutical composition to the subject. In another aspect, the at least one nAChR subtype is an α3β4 nAChR and the subject is a mammal such as, for example, a human, dog, cat, rat, mouse, guinea pig, non-human primate, rabbit, or horse. In still another aspect, the disease or disorder can be substance abuse, addiction, or any combination thereof.

### Pharmaceutical Compositions

In certain aspects, the disclosure provides a pharmaceutical composition comprising a therapeutically effective amount of a compound disclosed herein, and one or more pharmaceutically acceptable diluents, preservatives, solubilizers, emulsifiers, adjuvants, excipients, or carriers. The pharmaceutical composition can be used, for example, for treating substance abuse or addiction in a subject.

In certain aspects, the disclosure provides a pharmaceutical composition comprising the compounds of the disclosure together with one or more pharmaceutically acceptable excipients or vehicles, and optionally other therapeutic and/or prophylactic ingredients. Such excipients include liquids such as water, saline, glycerol, polyethylene glycol, hyaluronic acid, ethanol, and the like.

Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions, or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the disclosure can be administered as pharmaceutical formulations including those suitable for oral (including buccal and sub-lingual), rectal, nasal, topical, pulmonary, vaginal, or parenteral (including intramuscular, intra-arterial, intrathecal, subcutaneous, and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced elsewhere herein.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension, or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents, and suspending agents.

The sterile injectable formulation can also be a sterile injectable solution or a suspension in a nontoxic parenterally acceptable diluent or solvent. Among the acceptable vehicles and solvents that can be employed are water, Ringer's solution, and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as an continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing or wetting agents, and suspending agents. The sterile injectable formulation can also be a sterile injectable solution or a suspension in a nontoxic parenterally acceptable diluent or solvent. Among the acceptable vehicles and solvents that can be employed are water, Ringer's solution, and isotonic sodium chloride solution. In addition, sterile, fixed oils, fatty esters, or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions.

They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax, and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels, and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the disclosure, generally a therapeutic amount will be in the range of about 0.01 mg/kg to about 250 mg/kg body weight, more preferably about 0.1 mg/kg to about 10 mg/kg, in at least one dose. In larger mammals the indicated daily dosage can be from about 1 mg to 300 mg, one or more times per day, more preferably in the range of about 10 mg to 200 mg. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a substituent,” “a solvent,” or “an enantiomer,” includes, but is not limited to, mixtures or combinations of two or more such substituents, solvents, or enantiomers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “pharmaceutically acceptable vehicle” refers to a diluent, adjuvant, excipient, or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation.

“Pharmaceutically acceptable carriers” for therapeutic use are well known in the pharmaceutical art, and are described, for example, in Remington's Pharmaceutical Sciences, 18th Edition (Easton, Pennsylvania: Mack Publishing Company, 1990). For example, sterile saline and phosphate-buffered saline at physiological pH can be used. Preservatives, stabilizers, dyes, and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

“Pharmaceutically acceptable salts” refers to salts or zwitterionic forms of the present compounds. Salts of the present compounds can be prepared during the final isolation and purification of the compounds or separately by reacting the compound with an acid having a suitable cation. The pharmaceutically acceptable salts of the present compounds can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, tartaric, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides. In light of the foregoing, any reference to compounds of the present disclosure appearing herein is intended to include the present compounds as well as pharmaceutically acceptable salts thereof. “Pharmaceutically acceptable salt” refers to both acid and base addition salts.

“Modulating” or “modulate” refers to the treating, prevention, suppression, enhancement or induction of a function, condition, or disorder. For example, it is believed that the compounds of the present disclosure can modulate atherosclerosis by stimulating the removal of cholesterol from atherosclerotic lesions in a human.

“Treating” or “treatment” covers the treatment of a disease or disorder described herein, in a subject, preferably a human, and includes: (i) inhibiting a disease or disorder, i.e., arresting its development; (ii.) relieving a disease or disorder, i.e., causing regression of the disorder; (iii.) slowing progression of the disorder; and/or (iv.) inhibiting, relieving, or slowing progression of one or more symptoms of the disease or disorder

“Subject” refers to a warm blooded animal such as a mammal, preferably a human, or a human child, which is afflicted with, or has the potential to be afflicted with one or more diseases and disorders described herein.

### Chemical Residues and Substituents

Terms used herein may be preceded and/or followed by a single dash, “-,” or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)CC alkyl indicate the same functionality; similarly arylalkyl and -alkylaryl indicate the same functionality.

“Acetyl” means a —C(O)CH₃ group.

“Alkenyl” means a straight or branched chain hydrocarbon containing from 2 to 10 carbons, unless otherwise specified, and containing at least one carbon-carbon double bond.

Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

“Alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

“Alkyl” means a straight or branched chain hydrocarbon containing from 1 to 10 carbon atoms unless otherwise specified. Representative examples of alkyl include, but are not limited to, methyl, ethyl, n-propyl, iso-propyl, n-butyl, see-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

“Amino” means a group of formula —NR^{a}R^{b} wherein R^{a} and R^{b} are independently selected from hydrogen and C1-C4 alkyl.

“Cyano” and “nitrile” as used herein, mean a —CN group.

“Cycloalkyl” means a monocyclic or a bicyclic cycloalkyl ring containing from 3 to 8 carbon atoms, where such groups can be saturated or unsaturated, but not aromatic. In certain embodiments, cycloalkyl groups are fully saturated. In certain embodiments of the disclosure, the cycloalkyl is cyclopropyl, cyclobutyl, cyclopentyl, cyclohexyl, or cycloheptyl. In some aspects, two substituents in a compound of Formula I (e.g. R¹ and R²) can be connected by a bond or a C1-C4 carbon bridge to form a cycloalkyl group). For example, when R¹ and R² are connected by a single bond, a cyclopropyl group is formed.

“Halo” or “halogen” means —C, —Br, —I or —F. In certain embodiments, “halo” or “halogen” refers to —Cl or —F.

“Haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl. In certain embodiments, each “haloalkyl” is a fluoroalkyl, for example, a polyfluoroalkyl such as a substantially perfluorinated alkyl.

“Nitro” means a group of formula —NO₂.

“Saturated” means the referenced chemical structure does not contain any multiple carbon-carbon bonds. For example, a saturated cycloalkyl group as defined herein includes cyclohexyl, cyclopropyl, and the like.

“Unsaturated” means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

Many organic compounds exist in optically active forms having the ability to rotate the plane of plane-polarized light. In describing an optically active compound, the prefixes D and L or R and S are used to denote the absolute configuration of the molecule about its chiral center(s).

The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

A chiral center can be “substantially” (R) or “substantially” (S). In one aspect, a “substantially” (R) or (S) chiral center can be at least 95% (R) or 95% (S), or at least 99% (R) or 99% (S). In another aspect, a “substantially” (R) or (S) chiral center can be 100% (R) or 100% (S), respectively. Stereochemistry can be determined by any technique in the art including, but not limited to, crystallography, 2-dimensional NMR spectroscopy, or the like.

Unless otherwise specified, pressures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The following list of exemplary aspects supports and is supported by the disclosure provided herein.

Aspect 1. A compound of Formula I or a pharmaceutically acceptable salt thereof:

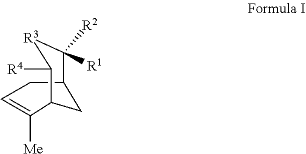

wherein R¹ and R² are independently selected from hydrogen, C1-C4 alkyl, C1-C4 haloalkyl, C3-C6 cycloalkyl, phenyl, and deuterated C1-C4 alkyl, or wherein R¹ and R² together are part of a cycloalkyl group;
wherein R³ is selected from NR⁵, S, and O;
   wherein R⁵ is selected from hydrogen, C1-C4 alkyl, and acetyl; and
wherein R⁴ is selected from (1) a C1-C4 alkyl or alkenyl group substituted with a substituted or unsubstituted aryl group or (2) a substituted or unsubstituted 5 to 10-membered aryl or heteroaryl group;
provided that when R¹ and R² are methyl and R³ is NH, R⁴ is not

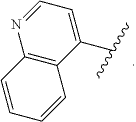 [SMILES: *c1ccnc2ccccc12]

Aspect 2. The compound of aspect 1, wherein R¹ and R² are independently selected from hydrogen, methyl, and ethyl.

Aspect 3. The compound of aspect 1 or 2, wherein R¹ and R² are methyl.

Aspect 4. The compound of aspect 1 or 2, wherein R¹ and R² together are part of a cyclopropyl group.

Aspect 5. The compound of any one of the preceding aspects, wherein R³ is NR⁵ and R⁵ is hydrogen, methyl, or ethyl.

Aspect 6. The compound of any one of aspects 1-4, wherein R³ is O.

Aspect 7. The compound of aspect 1, wherein R¹ and R² are methyl and R³ is NH.

Aspect 8. The compound of any one of aspects 1-7, wherein R⁴ is a phenyl group substituted with one or more of: hydroxyl, nitro, amino, halo, C₁-C₄ haloalkyl, C1-C₄ alkoxy, or C₁-C₄ alkyl, C3-C6 cycloalkyl, or any combination thereof.

Aspect 9. The method of any one of aspects 1-7, wherein R⁴ is a quinoline group substituted with one or more of: hydroxyl, nitro, amino, halo, C₁-C₄ haloalkyl, C₁-C₄ alkoxy, or C₁-C₄ alkyl, C3-C6 cycloalkyl, or any combination thereof.

Aspect 10. The compound of any one of aspects 1-7, wherein R⁴ is

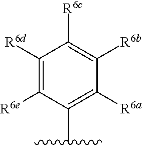

and wherein R⁶, R^{6b}, R^{6c}, R^{6d}, and R^{6e} are independently selected from hydrogen, halogen, hydroxyl, N-acetyl, —OCF₃, nitro, isopropyl, cyclopropyl, tert-butyl, dimethylamino, or any combination thereof.

Aspect 11. The compound of any one of aspect 1-7, wherein R⁴ is

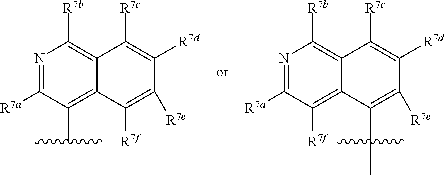

and wherein R^{7a}, R^{7b}, R^{7c}, R^{7d}, R^{7e}, and R^{7f} are independently selected from hydrogen, halogen, hydroxyl, N-acetyl, —OCF₃, nitro, isopropyl, cyclopropyl, tert-butyl, dimethylamino, or any combination thereof.

Aspect 12. The compound of any one of aspects 1-7, wherein R⁴ is

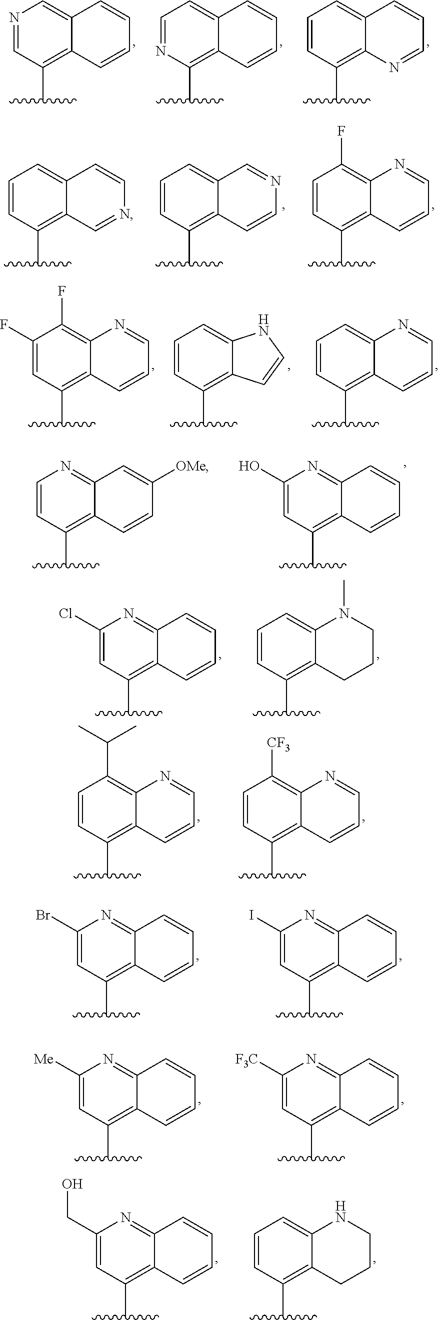

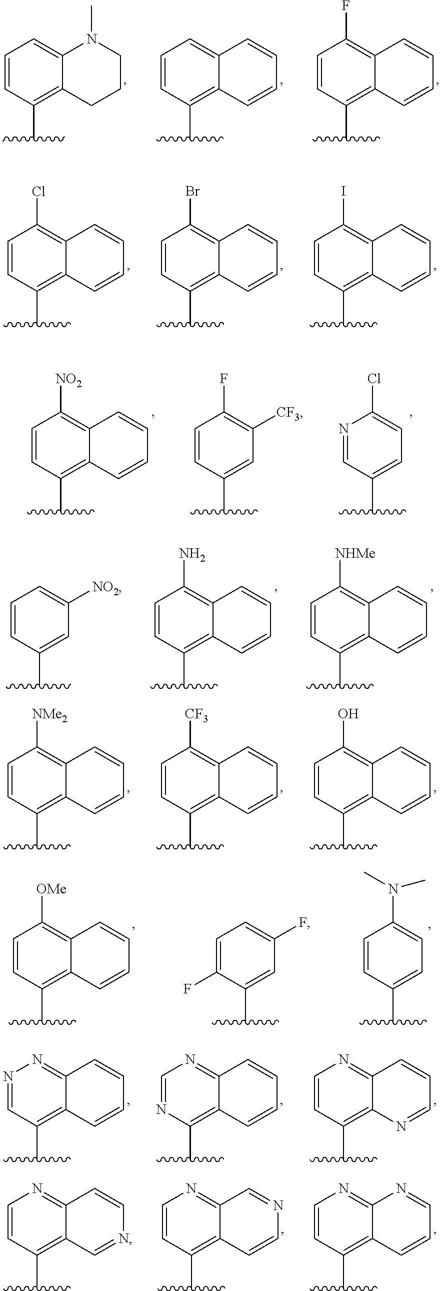

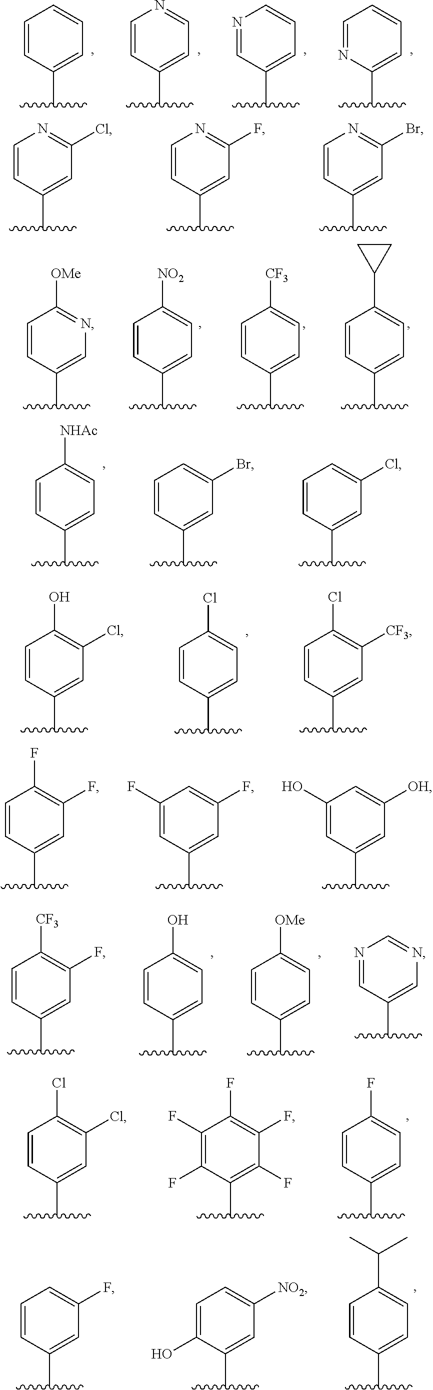

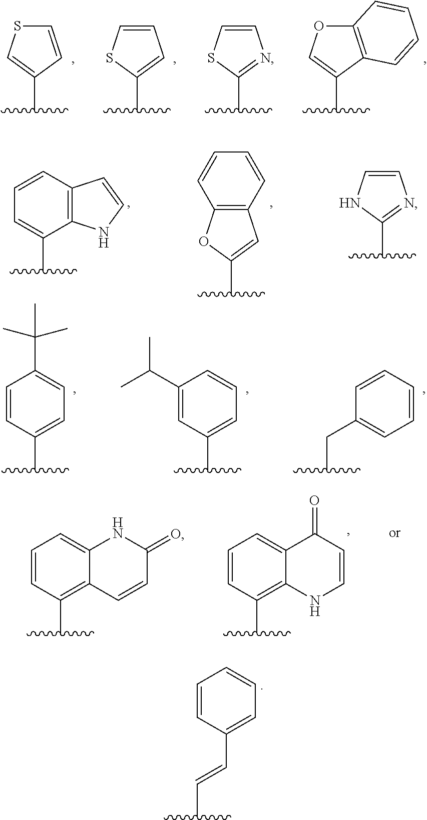

Aspect 13. The compound of aspect 1, wherein the compound of Formula I is selected from

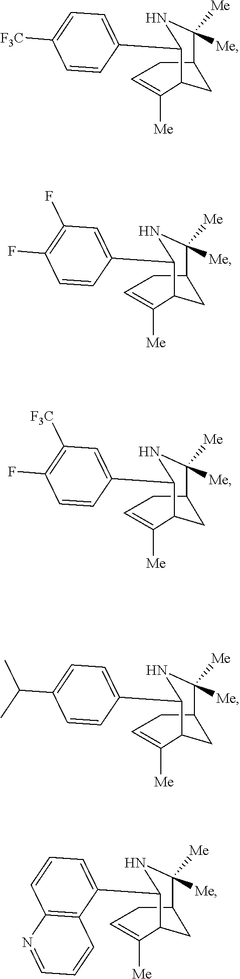

or a pharmaceutically acceptable salt thereof.

Aspect 14. The compound of aspect 1, wherein the compound of Formula I is selected from

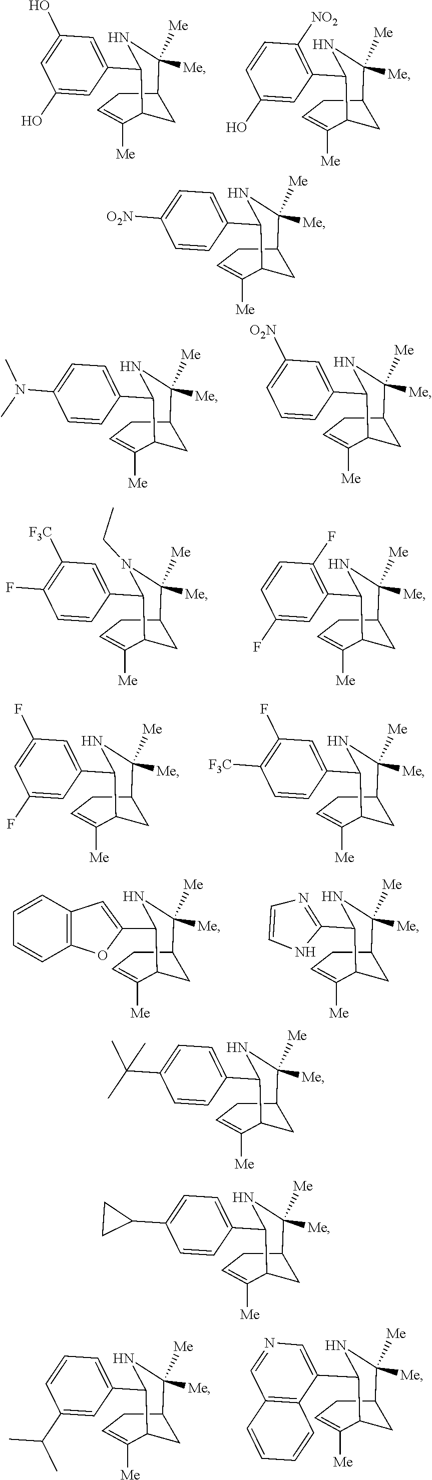

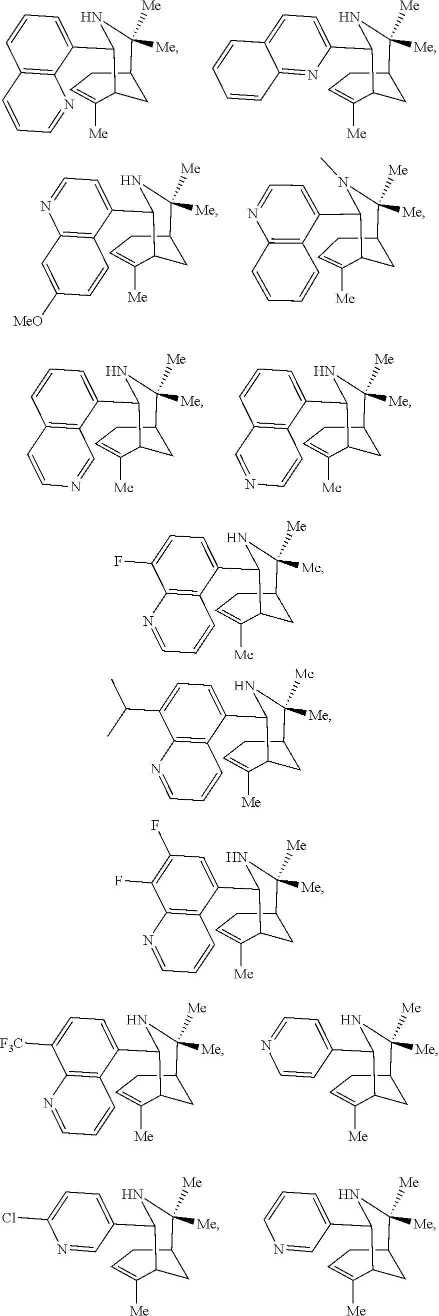

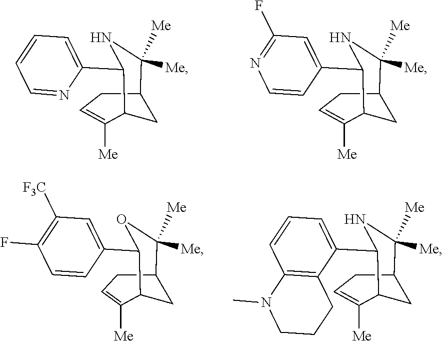

or a pharmaceutically acceptable salt thereof.

Aspect 15. The compound of aspect 1, wherein the compound of Formula I is selected from

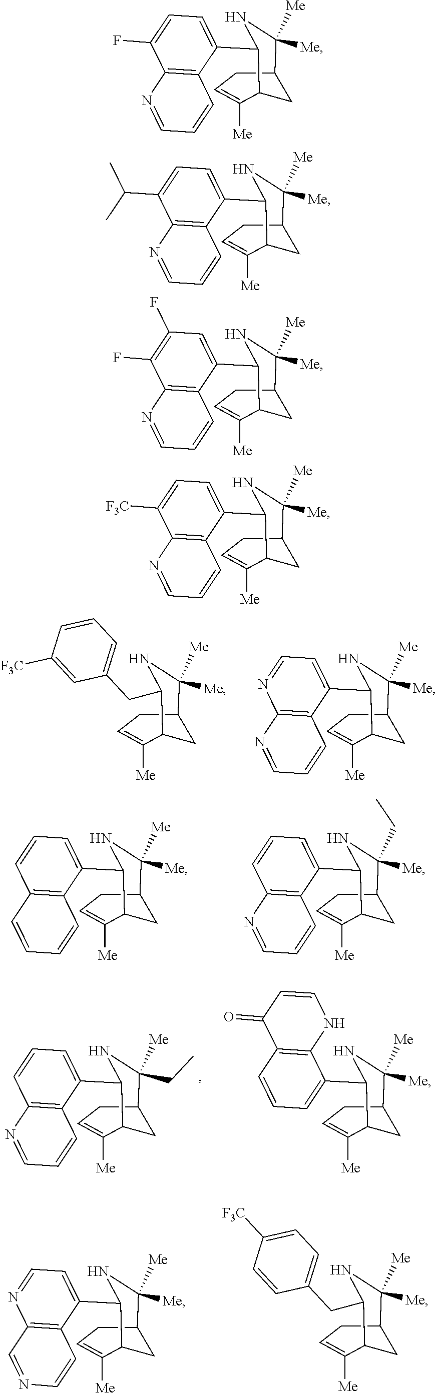

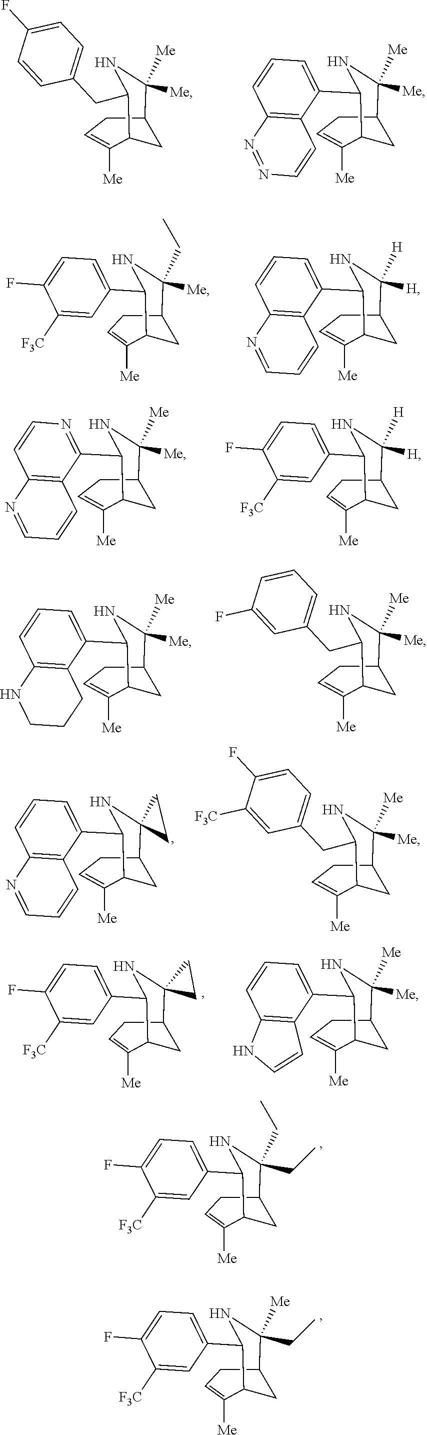

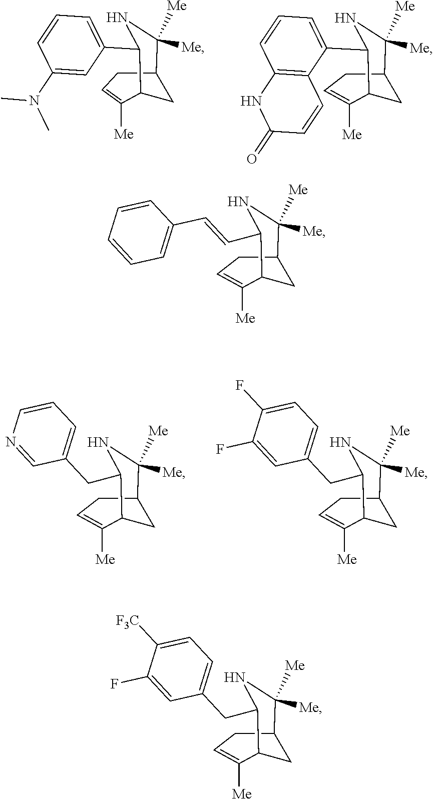

or a pharmaceutically acceptable salt thereof.

Aspect 16. The compound of any one of aspects 1-15, wherein the compound is a (+) enantiomer, a (−) enantiomer, or any combination thereof.

Aspect 17. The compound of any one of aspects 1-15, wherein the compound of Formula I has a chiral center a

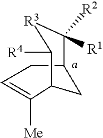

wherein the stereochemistry at chiral center a is substantially R, substantially S, or racemic.

Aspect 18. A method for inhibiting the activity of at least one nicotinic acetylcholine receptor (nAChR) subtype, the method comprising contacting the nAChR subtype with the compound of any one of the preceding aspects.

Aspect 19. The method of aspect 18, wherein the at least one nAChR subtype is an α3β4 nAChR.

Aspect 20. The method of aspect 18 or 19, wherein the compound has an IC₅₀ for the nAChR subtype of less than about 1 μM.

Aspect 21. The method of any one of aspects 18-20, wherein the compound has an IC₅₀ for the nAChR subtype of from about 0.25 to about 0.5 μM.

Aspect 22. A pharmaceutical composition comprising the compound of any one of aspects 1-17, an enantiomer thereof, or a pharmaceutically acceptable salt thereof, and a pharmaceutically acceptable carrier or excipient.

Aspect 23. A method for treating or preventing a disease or disorder associated with aberrant activity of at least one nicotinic acetylcholine receptor (nAChR) subtype in a subject, the method comprising administering a therapeutically effective amount of the compound of any one of aspects 1-17 or the composition of aspect 22 to the subject.

Aspect 24. The method of aspect 23, wherein the at least one nAChR subtype is an α3β4 nAChR.

Aspect 25. The method of aspect 23 or 24, wherein the subject is a mammal.

Aspect 26. The method of aspect 25 wherein the mammal is a human, dog, cat, rat, mouse, guinea pig, non-human primate, rabbit, or horse.

Aspect 27. The method of any one of aspects 23-26, where the disease or disorder comprises substance abuse, addiction, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Synthesis Strategy and Motivation

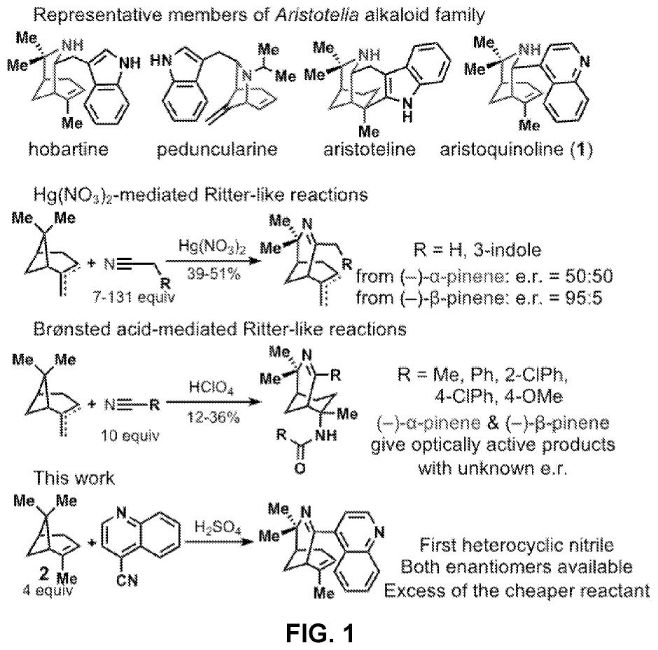

Several Aristotelia alkaloids have been synthesized by a variety of methods. The earliest of these approaches employed Hg(NO₃)₂-mediated Ritter-like reactions between α- or β-pinene and alkyl nitriles to generate the 3-azabicyclo[3.3.1]non-6-ene core (FIG. 1). When (−)-β-pinene was employed, these reactions were highly stereospecific, whereas (−)-α-pinene (2) yielded racemic products. Applying this strategy to the synthesis of both enantiomers of 1 is problematic, given the limited availability of (+)-β-pinene. Later studies did establish that Brønsted acids induced similar Ritter-like reactions, with both α- and β-pinene producing optically active products. However, with Brønsted acids, amides arising from a second Ritter reaction were formed as the major products (FIG. 1). Moreover, the enantiopurity of these products was not reported. In addition to the stereochemical issues, the established Ritter-like reactions have suffered from the need for large excesses of the nitrile and have not been explored to introduce heterocycles like quinolines to the monoterpene core. Thus, the Ritter-like reaction was leveraged to provide rapid access to both enantiomers of 1 (FIG. 1).

The reaction of 2 and 4-cyanoquinoline (3) in the presence of H₂SO₄ produced the intended product 4 as the major product alongside an additional isomeric imine (Scheme 1). A comparison of the corresponding ¹H and ¹³C NMR spectra revealed a striking similarity between the two products, except for the geminal methyl groups, which appeared as a pair of singlets in 4 and a pair of doublets in 5. COSY correlations in 5 indicated these methyl groups are contained in an isopropyl group adjacent to a quaternary carbon. Further analysis of the HMBC and COSY spectra revealed the imine nitrogen of the minor product was bound to the cyclohexene ring, consistent with the structure of 5. Reduction of 4 with NaB(OAc)₃H delivers a hydride to the less hindered face of the imine, producing a single diastereomer whose ¹H and ¹³C spectra are consistent with naturally occurring 1 and (−)-1 whose structure was confirmed by X-ray crystallography (vide infra). A similarly diastereoselective reduction of 5 yielded isoaristoquinoline (6).

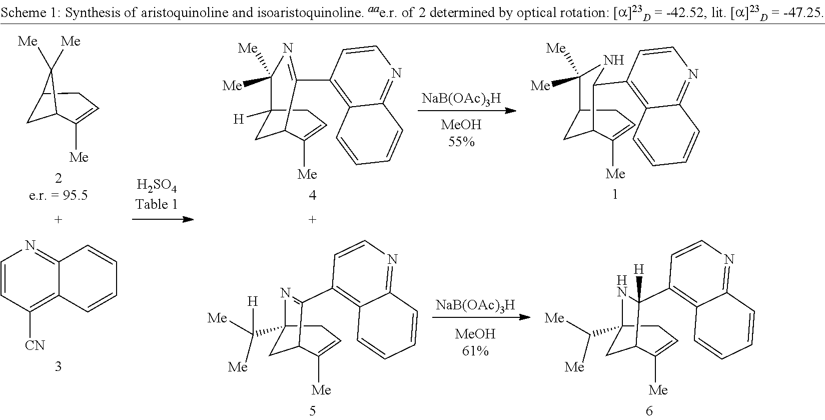

Encouraged by these initial results, reaction conditions were developed that would favor the selective formation of 4 (Table 1). The yields of 4 and 5 were minimally impacted when toluene was used in place of benzene, whereas the use of polar solvents such as acetic acid, DMF and DMSO proved detrimental to the reaction (entries 1-5). Replacing H₂SO₄ with other Brønsted acids resulted in no observed product formation (entry 6). Neither cooling nor heating the reactions led to substantial changes in the product yields (entries 7-8). Conducting the reactions with an excess of α-pinene 2 did improve the yields of imine 4, whereas increasing the relative amounts of nitrile 3 led to a slight increase in the undesired product 5 (entries 9-10).

TABLE 1
Optimization of Ritter-Like Reaction Conditions

| entry | 2:3 | solvent | temp | % Yield 4^{a} | % Yield 5^{a} |
|---|---|---|---|---|---|
| 1 | 1:1 | benzene | rt | 21 | 3 |
| 2 | 1:1 | toluene | rt | 23 (20) | 4 (4) |
| 3 | 1:1 | AcOH | rt | 0 | 0 |
| 4 | 1:1 | DMF | rt | 0 | 0 |
| 5 | 1:1 | DMSO | rt | 0 | 0 |
| 6^{b} | 1:1 | toluene | rt | 0 | 0 |
| 7 | 1:1 | toluene | −10° C. | 19 | 3 |
| 8 | 1:1 | toluene | 110° C. | 15 | 3 |
| 9 | 1:4 | toluene | rt | 23 | 8 |
| 10 | 4:1 | toluene | rt | 35 | 7 |
| 11^{c} | 4:1 | toluene | rt | 29 | 5 |
| 12 ^{d} | 4:1 | toluene | rt | 41 | 6 |

^{a}Yields determined by ¹H NMR. Isolated yields in parentheses.
^{b}HBF₄•OEt₂, TFA, or polyphosphoric acid used instead of H₂SO₄.
^{c}7 used in place of 2.
^{d} 8 used in place of 2.

Finally, both 4 and 5 were produced in similar yields when (−)-limonene (7) and (−)-α-terpineol (8) were used in place of 2 (entries 11-12). Interestingly, under all the reaction conditions tested, no evidence of products arising from a second Ritter reaction was observed via LC/MS or ¹H NMR analysis of the crude reaction mixtures. With these optimized conditions, the yield of 4 is comparable to that observed with previous studies; however, these conditions avoid the use of large excess of the nitrile, and instead rely on an excess of the abundant, inexpensive terpenes.

While these conditions allowed for a convergent synthesis of 1, the products from these reactions were nearly racemic. Notably, terpenes 2, 7, and 8 all resulted in products with similarly low levels of enantiopurity. Given this surprising departure from previous Brønsted-acid catalyzed Ritter reactions, the reaction mechanism was investigated in hopes of being able to design a more stereoselective reaction. The formation of 4 and 5 is consistent with the mechanism proposed in Scheme 2A. In the presence of acid, 2, 7 and 8 generate the same carbocation intermediate 9. Nucleophilic attack of the carbocation of intermediate 9 by 3 in a Ritter-like reaction forms a nitrilium ion that is subsequently intercepted by the endocyclic olefin, ultimately giving rise to 4. Conversely, if 9 undergoes a 1,2-hydride shift to form 10 prior to attack by the nitrile, imine 5 is produced. Intermediate 10 may also be formed from the deprotonation of 9 to 11, and subsequent reprotonation of the tetrasubstituted olefin. The low enantiomeric ratio of 4 suggests the conversion of 9 to the achiral intermediates 10 or 11 is reversible. Alternatively, the stereochemical scrambling of the products may arise from the reversible protonation of the endocyclic olefin to generate an achiral carbocation intermediate.

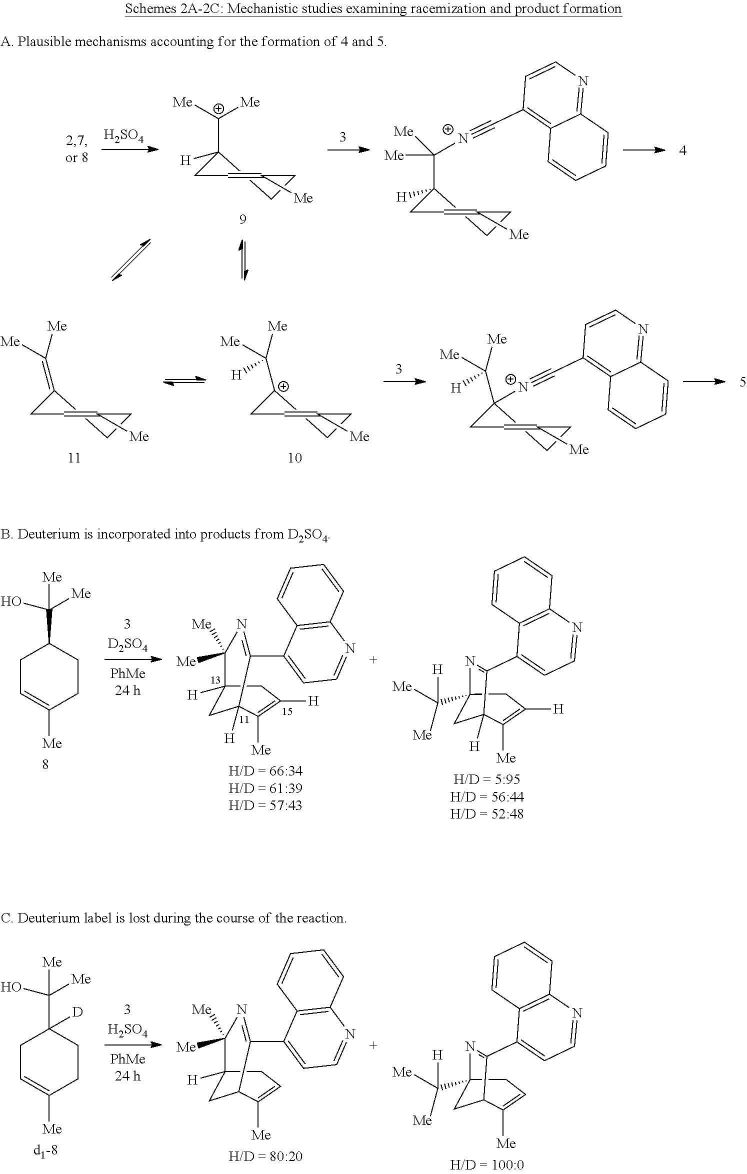

To investigate these possible mechanisms, two isotopic labeling studies were conducted. First, the reaction between 3 and 8 was conducted in the presence of D₂SO₄ (Scheme 2B). If intermediate 11 is formed during the reaction, products 4 and 5 would be expected to incorporate a deuterium atom at C13. Indeed, under these conditions compounds 4 and 5 are 34% and 95% deuterated at these positions, clearly showing intermediate 11 is formed in the reaction. To confirm that a concerted 1,2-hydride shift does not occur, deuterium-labeled terpineol (di-8) was synthesized from the reduction of terpinolene oxide with LiAID4. Reacting d1-8 with 3 in the presence of H₂SO₄ produced 4 and 5 with 80% and 100% loss of deuterium at C13, respectively. (Scheme 2C). Taken together, these studies reveal that the interconversion of 9 and 10 occurs through the generation of intermediate 11.

With these mechanistic understandings, it is likely the modest yields of 4 are due to the poor nucleophilicity of 3, particularly under the acidic reaction conditions that lead to the protonation of the quinoline nitrogen. Notably, under similar reactions aryl nitriles that lack a basic nitrogen do not produce isomeric products like 5. This finding suggests the reduced nucleophilicity of 3 also contributes to the formation of 5, by slowing the production of 4 such that the conversion from 9 to 10 is competitive. The low nucleophilicity of 3 may also explain why products arising from the second Ritter reaction were not observed. Regarding the stereoselectivity of the transformation, the incorporation of deuterium at C11 and C15 of 4 and 5 in the presence of D₂SO₄ (Scheme 2B) indicates that protonation of the endocyclic olefin also contributes to the racemization of the products. This suggests that Brønsted acid-promoted Ritter-reactions are unlikely to yield 4 with high levels of enantiopurity.

In light of these considerations, it was reasoned that reversing the roles of the nucleophile and electrophile could improve both the yield and stereoselectivity of the reaction. To test this hypothesis, an alternative approach was adopted, employing an aza-Prins reaction as the key cyclization event (Scheme 3). The tertiary alcohol of (−)-8 was first converted to the corresponding azide 12. Subsequent reduction of 12 with zinc provided the primary amine (−)-13. Condensation of (−)-13 and 4-quinolinecarboxaldehyde (14) in the presence of 3 Å molecular sieves produced the imine 15. Although 15 proved difficult to isolate, the addition of a solution of 15 generated in situ to TFA led to the rapid and exclusive formation of 1.

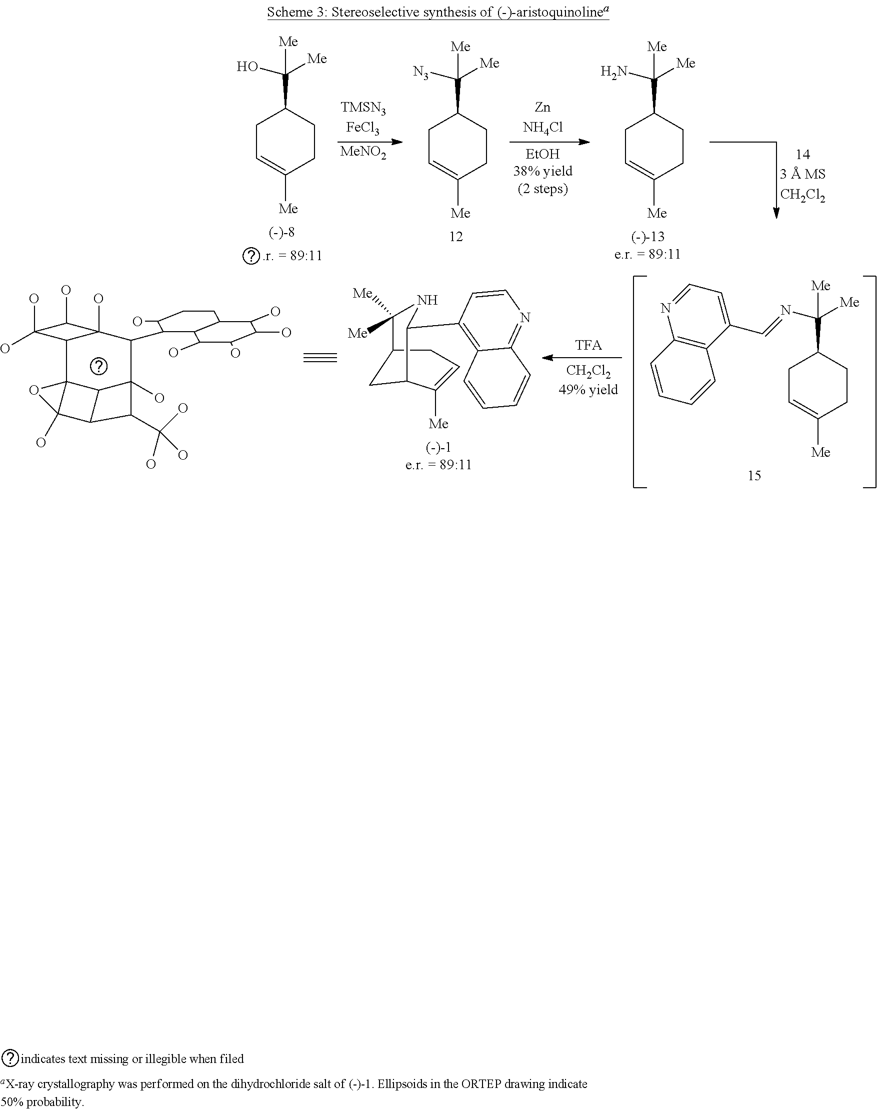

Compound 1 formed through this route possessed considerably improved enantiopurity (e.r.=89(−):11(+)) compared to the Ritter-like reaction product. A closer inspection of (−)-8 by optical rotation and (−)-13 by conversion to its corresponding (R)-Mosher amide revealed the intermediates possessed similar levels of enantiopurity, indicating the aza-Prins reaction proceeds with complete stereospecificity. Using this enantioenriched material, the configuration of the C9 stereogenic center and absolute configuration of (−)-1 was determined through single-crystal X-ray diffraction. In this X-ray crystal structure, the aromatic quinoline ring is positioned directly over the C17 methyl group, accounting for its considerable upfield shift in the ¹H NMR spectrum (δ=0.66 ppm). Notably, the structure proposed by Arias places the quinoline distal to the C17 methyl group and is unlikely to produce the observed anisotropic effect. Finally, adopting an identical procedure, (+)-8 was successfully converted to (+)-1 in 13% overall yield and high enantiopurity (e.r. s 1(−):99(+)).

With both enantiomers of 1 in hand, their activities at the rat α3β4 nAChRs were evaluated by employing a cell-based Ca²⁺ influx assay (Table 2). As expected, neither enantiomer of 1 nor racemic 6 produced any activation of the nAChRs at concentrations up to 100 μM. However, 1 and 6 effectively inhibited the action of (±)-epibatidine with similar potency to the pan-nAChR channel-blocker (±)-mecamylamine. Interestingly, (+)-1 antagonizes the α3β4 nAChRs more potently than (−)-1, and is nearly identical to the IC₅₀ reported for 1 isolated from A. chilensis, strongly implying that (+)-1 is the naturally occurring enantiomer.

TABLE 2
Activity at the Rat α3β4 nAChRs
Determined by Calcium Influx Assay

| Compound | e.r.^{a} | EC₅₀(μM)^{b} | Eₘₐₓ(%)^{c} | IC₅₀ (μM)^{d} |
|---|---|---|---|---|
| (±)-1 | 51:49 | — | <5 | 3.5 ± 0.9 |
| (−)-1 | 89:11 | — | <5 | 3.4 ± 1.2 |
| (+)-1 | 1:99 | — | <5 | 0.89 ± 0.36 |
| 6 | 50:50 | — | <5 | 3.4 ± 0.6 |
| MEC^{e} | 50:50 | — | <5 | 1.1 ± 0.1 |
| EPI^{f} | 50:50 | 0.028 ± 0.006 | 100 | 0.012 ± 0.001 |

^{a}Ratios of (−)-1:(+)-1 and (−)-6:(+)-6 determined by chiral HPLC.
^{b}Half maximal effective concentration. EC₅₀ values are not reported for compounds eliciting <5% receptor activation.
^{c}Maximal effective concentration relative to the maximal effect elicited by the agonist (±)-epibatidine.
^{d}Half maximal inhibitory concentration for inhibition of receptor activation evoked by 100 nM (±)-epibatidine.
^{e}(±)-mecamylamine.
^{f}(±)-epibatidine.

In conclusion, two approaches to 1 have been investigated, resulting in the first synthesis of the alkaloid. By studying the mechanism behind the Ritter-like reaction, it was determined that this classical approach is unlikely to lead to enantioenriched 1. The second strategy to form the 3-azabicyclo[3.3.1]non-6-ene via an aza-Prins cyclization led to 1 with good enantiopurity, which enabled us to unambiguously establish the absolute and relative configuration via X-ray crystallography. With access to both enantiomers of 1, it has been identified that (+)-1 possesses considerably greater inhibitory activity at the α3β4 nAChR than its antipode. While the activity observed is similar to that reported for the isolated alkaloid, the configuration of (+)-1 is opposite that reported for other Aristotelia alkaloids including those found in A. chilensis. This unexpected result indicates that in addition to being unique as the sole quinoline-containing Aristotelia alkaloid, 1 also appears to be the only member of its enantiomeric series. In addition to these naturally occurring alkaloids, the novel isoaristoquinoline 6 bearing a 6-azabicyclo[3.2.1]oct-2-ene scaffold reminiscent of peduncularine, was also shown to possess inhibitory activity and thus represents the first entry into a previously unknown class of nAChR ligands.

### Example 2: General Procedures

All reactions were conducted under a nitrogen atmosphere in oven-dried round bottom flasks fitted with rubber septa. All reagents and solvents were purchased from commercial suppliers and used without further purification. The reactions were monitored using Macherey-Nagel silica gel 60 F254 TLC plates and visualized using a UV Lamp (254 nm), 12 chamber, and/or Dragendorff's reagent. Compounds were purified by column chromatography using silica gel from Machery-Nagel mesh size 40-63 μm. Prior to biological evaluation compounds (±)-1, (+)-1, (−)-1, and (±)-6 were converted to hydrochloride salts by careful addition of ethereal HCl to solutions of the corresponding free base in ether. The purity of the HCl salts was determined on an Agilent 1260 Infinity II fitted with a Phenomenex Luna Omega PS-C18 column (100×4.6 mm²) and a DAD detector. A gradient of acetonitrile/water (20-45%) with 0.1% formic acid with a flow rate of 1 mL/min was used. The purity of all samples tested in the assay was determined to be >95% by HPLC. The enantiomeric excess of compounds was determined using the above-mentioned HPLC instrument fitted with a Reflect-I Amylose A 5 μm column (15 cm×4.6 mm) and a DAD detector. The samples were analyzed using an isocratic elution with 5% EtOH in hexanes with a flow rate of 0.8 mL/min. The samples were prepared in 5% EtOH in hexanes with 0.1% triethylamine. ¹H and ¹³C NMR were recorded on a Bruker AVIII 400 MHz spectrometer and referenced to the residual solvent signal (¹H δ=7.26, ¹³C δ=77.16 ppm). High-resolution mass spectroscopy (HRMS) was performed on a Waters Synapt G2-Si ESI mass spectrometer.

### Example 3: Optimization of Ritter-Like Reaction

Initial conditions: Concentrated sulfuric acid (0.03 mL, 0.55 mmol) was added dropwise to a stirred solution of 4-cyanoquinoline (10 mg, 0.065 mmol) in anhydrous toluene (4 mL) under an atmosphere of N₂ at 0° C., after which a solution of (−)-α-pinene (8.8 mg, 0.065 mmol) in anhydrous benzene (1 mL) was added. The reaction was allowed to warm to room temperature and stirred for 24 h. The reaction was diluted by the careful addition of H₂O (5 mL) and extracted with Et₂O (4×10 mL). The aqueous layer was basified with aqueous NaOH (5 M) to pH>10 and extracted with CH₂Cl₂ (4×10 mL). The organic layers were combined, dried over sodium sulfate, filtered, and concentrated in vacuo to give a crude residue. The sample was prepared for qNMR analysis by adding ethylene carbonate (1.00 mg) to a solution of the crude residue in 0.6 mL CDCl₃. ¹H NMR was performed at 400 MHz with 32 scans and D1=30 seconds. Characteristic peaks correlating to 4 and 5 were identified and integrated in respect to the internal standard to determine percent yield.

Modifications: Changes from the initial conditions detailed in Table 1 followed the same protocol as above, with modification to the indicated variable (i.e. solvent, acid, temperature, monoterpene, or stoichiometry).

### Example 4: Synthesis and Characterization of Compounds

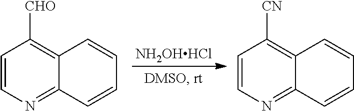

4-cyanoquinoline, 3: Hydroxylamine hydrochloride (0.490 g, 7.05 mmol) was added portion-wise to a stirred solution of quinoline-4-carboxaldehyde (1.00 g, 6.36 mmol) in anhydrous DMSO (4 mL) under an atmosphere of N₂ at room temperature. The reaction was allowed to stir at room temperature for 48 h and subsequently quenched by the addition of H₂O (30 mL) and extracted with Et₂O (4×30 mL). The organic layers were combined, dried over magnesium sulfate, filtered, and concentrated in vacuo to give a crude solid which was purified by silica gel chromatography (CH₂Cl₂:MeOH 100:0 to 80:20) to give 3 as a light orange-colored solid (0.640 g, 65% yield). The ¹H and ¹³C-NMR are consistent with those reported in literature.

Generic Scheme for Forming Bicyclic Molecules: A generic scheme for forming a bicyclic molecule having (+) stereochemistry is provided in Scheme 4 below. Additional specific synthetic mechanisms are also disclosed herein.

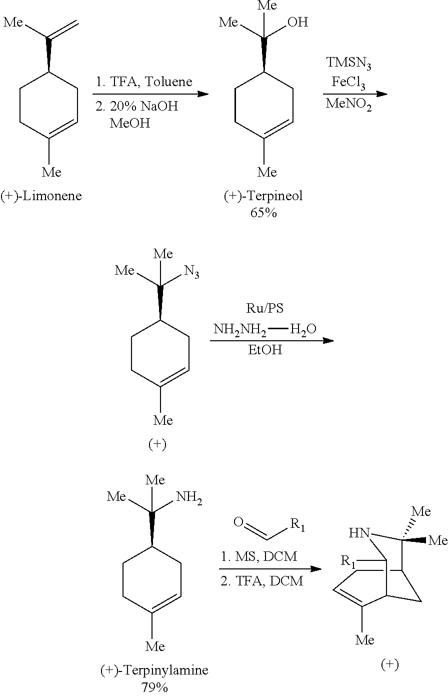

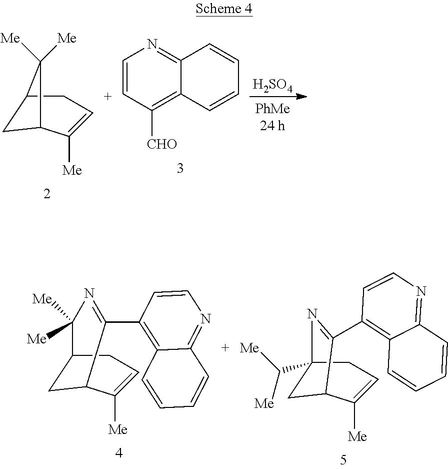

Ritter-Like Reaction: A solution of (−)-α-pinene (179 mg, 1.31 mmol) in anhydrous toluene (1 mL) was added dropwise to a stirred solution of sulfuric acid (18 M, 2.8 mL, 50.4 mmol) and quinoline-4-carbonitrile (200 mg, 1.30 mmol) in anhydrous toluene (4 mL) under an atmosphere of N₂ at 0° C. The reaction was allowed to warm to room temperature and stirred for 24 h. The reaction was diluted by the careful addition of H₂O (5 mL) at 0° C. (ice-bath) and extracted with Et₂O (2×50 mL). The aqueous layer was basified with aqueous NaOH (10% w/v) to pH=14 and extracted with CH₂Cl₂ (3×30 mL). The CH₂Cl₂ layers were combined, dried over sodium sulfate, filtered, and concentrated in vacuo to give a crude residue, which was purified using silica gel chromatography (hexanes:EtOAc, 100:0 to 0:100) to give 4 (76 mg; 20% yield) and 5 (17 mg; 4.5% yield) as colorless oils.

Compound 4: ¹H NMR: (400 MHz, CDCl₃) δ 8.91 (d, J=4.4 Hz, 1H), 8.10 (dd, J=8.4, 1.3 Hz, 1H), 7.85 (dd, J=8.4, 1.4 Hz, 1H), 7.69 (ddd, J=8.4, 6.8, 1.5 Hz, 1H), 7.51 (ddd, J=8.3, 6.8, 1.3 Hz, 1H), 7.30 (d, J=4.4 Hz, 1H), 5.44 (dtd, J=2.9, 2.9, 1.5 Hz, 1H), 3.12 (t, J=3.3 Hz, 1H), 2.41 (m, 1H), 2.31 (m, 1H), 2.23 (dt, J=12.1, 2.7 Hz, 1H), 2.08 (m, 1H), 1.83 (dt, J=12.1, 2.8 Hz, 1H), 1.39 (s, 3H), 1.36 (s, 3H), 1.23 (m, 3H). ¹³C NMR: (101 MHz, CDCl₃) δ 166.6, 150.0, 149.0, 148.4, 134.4, 129.9, 129.6, 127.2, 125.7, 125.2, 122.7, 119.1, 60.4, 41.1, 33.8, 31.6, 29.0, 27.9, 25.7, 23.4. HRMS (ESI-TOF) m/z: [M+H]⁺ Calcd for C₂₀H₂₃N₂: 291.1861, found: 291.1859 (0.7 ppm). [α]²⁵_{D}: −1.98 (c 1.11, CHCl₃).

Compound 5: ¹H NMR: (400 MHz, CDCl₃) δ 8.95 (d, J=4.4 Hz, 1H), 8.58 (dd, J=8.6, 1.4 Hz, 1H), 8.12 (dd, J=8.6, 1.3 Hz, 1H), 7.73 (ddd, J=8.4, 6.8, 1.4 Hz, 1H), 7.58 (ddd, J=8.3, 6.8, 1.3 Hz, 1H), 7.47 (d, J=4.4 Hz, 1H), 5.28 (m, 1H), 3.37 (d, J=4.9 Hz, 1H), 2.46 (m, 1H), 2.08 (m, 3H), 1.80 (d, J=10.2 Hz, 1H), 1.64 (d, J=1.9 Hz, 3H), 1.14 (d, J=6.8 Hz, 3H), 1.08 (d, J=6.8 Hz, 3H). ¹³C NMR: (101 MHz, CDCl₃) δ 174.7, 149.9, 149.0, 140.3, 136.2, 129.9, 129.6, 127.6, 126.5, 126.0, 121.0, 120.6, 77.0, 52.3, 39.8, 35.0 30.1, 23.0, 17.9, 17.3. HRMS (ESI-TOF) m/z: [M+H]⁺ Calcd for C₂₀H₂₃N₂: 291.1861, found: 291.1855 (2.1 ppm). [α]²⁵_{D}: 0.0 (c 1.34, CHCl₃).

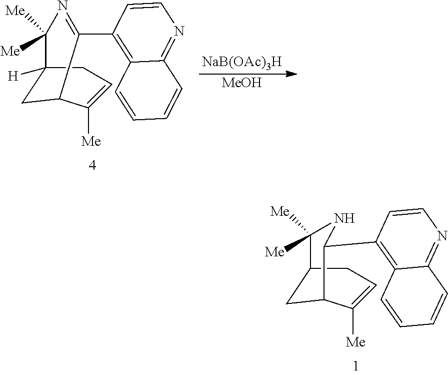

(±)Aristoquinoline; (±1): Sodium triacetoxyborohydride (176 mg, 0.830 mmol) was added portion-wise to a solution of 4 (40.0 mg, 0.138 mmol) in CH₂Cl₂ (6 mL) at room temperature and under an atmosphere of N₂. The reaction was allowed to stir at room temperature for 24 h and quenched by the addition of aq. NaHCO₃ (10 mL). The reaction mixture was concentrated in vacuo and the aqueous residue was extracted with CH₂Cl₂ (3×10 mL). The combined organic layers were dried over magnesium sulfate, filtered, and concentrated in vacuo to yield a crude residue that was purified by silica gel chromatography (CH₂Cl₂:MeOH, 100:0 to 80:20) to yield 1 as a colorless oil (22 mg, 55% yield).

¹H NMR: (400 MHz, CDCl₃) δ 8.79 (d, J=4.6 Hz, 1H), 8.11 (d, J=8.4 Hz, 1H), 8.07 (d, J=8.4 Hz, 1H), 7.69 (t, J=7.6 Hz, 1H), 7.58 (d, J=7.6 Hz, 1H), 7.19 (d, J=4.6 Hz, 1H), 5.57 (s (broad), 1H), 4.96 (d, J=2.4 Hz, 1H), 2.61 (q, J=3.0 Hz, 1H), 2.47 (dd, J=12.5, 3.2 Hz, 1H), 2.32 (m, 1H), 2.13 (m, 1H), 1.76 (dt, J=12.6, 3.3 Hz, 1H), 1.59 (dt, J=6.4, 3.2 Hz, 1H), 1.38 (s, 3H), 1.27 (s, 3H), 0.66 (d, J=2.5 Hz, 3H). ¹³C NMR: (101 MHz, CDCl₃) δ 150.2, 149.2, 148.2, 132.8, 130.6, 128.9, 126.4, 126.2, 125.0, 123.1, 117.4, 54.4, 53.2, 40.7, 34.6, 30.3, 29.4, 27.8, 25.7, 24.2. HRMS (ESI-TOF) m/z: [M+H]⁺ Calcd for C₂₀H₂₅N₂: 293.2018, found: 293.2006 (4.1 ppm). [α]²⁵_{D}=−2.31 (c 1.04, CHCl₃). Chiral HPLC: 8.872 min (49%), 10.344 (51%). RP-HPLC: 1.783 min (95%).

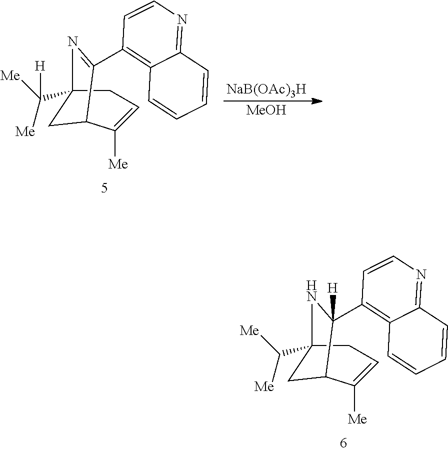 [SMILES: "MeOH" ; CC1=CC[C@@]2(C(C)C)CC1C(c1ccnc3ccccc13)=N2 ; CC1=CC[C@@]2(C(C)C)CC1[C@@H](c1ccnc3ccccc13)N2 ; "NaB(OAc)3H"]

Isoaristoquinoline; (±)-6: Sodium triacetoxyborohydride (132 mg, 0.623 mmol) was added portion-wise to a solution of 5 (30.0 mg, 0.103 mmol) in CH₂Cl₂ (4 mL) at room temperature. The reaction was allowed to stir at room temperature for 24 h and quenched by the addition of aq. NaHCO₃ (10 mL). The reaction mixture was concentrated in vacuo and the aqueous residue was extracted with Et₂O (3×20 mL). The combined organic layers were dried over magnesium sulfate, filtered, and concentrated in vacuo to yield a crude residue that was purified by silica gel chromatography (CH₂Cl₂:MeOH, 100:0 to 80:20) to yield 6 as a colorless oil (19 mg, 63% yield).

¹H NMR: (400 MHz, CDCl₃) δ=8.85 (d, J=4.5, 1H), 8.10 (ddd, J=9.9, 8.4, 1.4, 2H), 7.76 (dd, J=4.5, 0.9, 1H), 7.68 (ddd, J=8.4, 6.8, 1.4, 1H), 7.54 (ddd, J=8.3, 6.8, 1.4, 1H), 5.43 (d, J=4.5, 1H), 5.26 (d, J=4.5, 1H), 2.89-2.83 (m, 1H), 2.34 (dt, J=17.1, 2.5, 1H), 2.14-2.03 (m, 1H), 2.01-1.92 (m, 2H), 1.84 (hept, J=6.8, 1H), 1.25 (s, 1H), 1.06 (d, J=6.8, 3H), 1.02 (d, J=6.9, 3H), 0.64 (q, J=2.0, 3H). ¹³C NMR: (101 MHz, CDCl₃) δ=150.6, 148.5, 147.8, 136.9, 130.4, 128.7, 126.2, 122.7, 120.3, 118.8, 65.0, 63.9, 47.4, 41.6, 37.8, 37.7, 29.9, 23.0, 18.6, 17.8. HRMS (ESI-TOF) m/z: [M+H]⁺ Calc for C₂₀H₂₅N₂: 293.2018, found: 293.2007 (3.8 ppm). [α]²⁵_{D}=−0.26 (c 1.54, CHCl₃). Chiral HPLC: 8.911 min (50%), 10.392 min (50%). RP-HPLC: 2.088 min (97%).

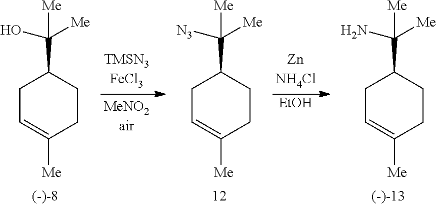 [SMILES: "EtOH" ; CC1=CC[C@@H](C(C)(C)O)CC1 ; CC1=CC[C@@H](C(C)(C)N=[N+]=[N-])CC1 ; CC1=CC[C@@H](C(C)(C)N)CC1]

Modified Dryzhakov Procedure: Using a modified procedure of Dryzhakov et al., trimethylsilyl azide (5.2 mL, 39.70 mmol) was added dropwise to a solution of (−)-α-terpineol ((−)-8; 2.00 g, 12.96 mmol) in nitromethane (6 mL) at room temperature and under air. To this mixture, FeCl₃ (105.4 mg, 0.65 mmol) was added portion-wise and the reaction was allowed to stir at room temperature for 40 minutes. The reaction was concentrated in vacuo to give a residue that was filtered through a silica plug using hexanes and concentrated in vacuo to yield 12 as a clear oil (2.00 g, 86% yield). The H NMR of the product was consistent with that reported in the literature² and was used without further purification.

Ammonium chloride (1.39 g, 26.00 mmol) was added portion-wise to a solution of 12 (1.90 g, 10.59 mmoles) in H₂O (10 mL) and EtOH (30 mL) at room temperature and under air. To this mixture, zinc powder (0.92 g, 14.07 mmoles) was added portion-wise and the reaction was allowed to stir at room temperature. After 30 minutes, the reaction mixture was filtered through a cotton plug and extracted with Et₂O (3×50 mL). The combined organic layers were dried over potassium carbonate and concentrated in vacuo to give the crude residue, which was purified using silica gel chromatography (CH₂Cl₂:MeOH:NH₄OH, 90:5:5) to yield (−)-13 as a clearoil (0.750 g, 44% yield). The ¹H and ¹³C NMR of the resulting product were consistent with those reported in the literature.

Mosher Amide Analysis: (R)-MTPA-Cl (7.5 μL, 0.03 mmol) was added to a stirred solution of (+)-8 (5 mg, 0.03 mmol) and DIPEA (8 μL, 0.05 mmol) in anhydrous CH₂Cl₂ (1 mL) at room temperature and under an atmosphere of N₂. The reaction was allowed to stir at room temperature for 20 h and quenched by the addition of NH₄Cl (1 mL) and extracted with CH₂Cl₂ (2×2 mL). The combined organic phase was dried over sodium sulfate, filtered, and concentrated in vacuo to yield a crude residue. The residue was purified by silica-gel column chromatography (hexanes:EtOAc 100:0 to 90:10) to yield the (R)-Mosher amides of 8. The d.r. for the amides was analyzed by integration of the ¹H-NMR shifts at 3.16 ppm and 3.20 ppm.

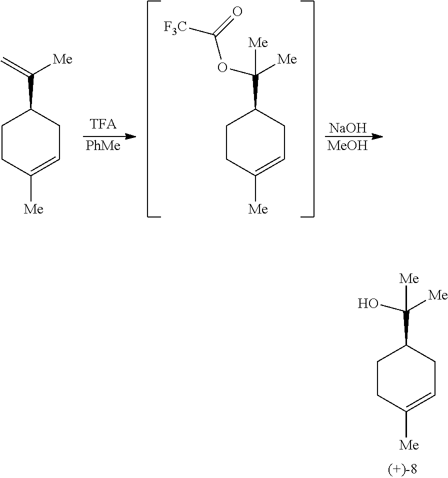

(+)-Terpineol; (+)-8: Using a modified procedure of Yuasa and Yuasa, trifluoroacetic acid (3.2 mL, 41.8 mmol) was added dropwise to a stirred solution of (+)-limonene (5.00 g, 36.7 mmol) in anhydrous toluene (12 mL) over 4 h at room temperature. The reaction mixture was allowed to stir for an additional 30 min and quenched by the addition of H₂O (20 mL). The separated organic layer was extracted with 5% NaHCO₃ (20 mL) and then with 5% NaCl (20 mL). The organic layer was concentrated in vacuo to yield a residue, which was dissolved in MeOH (10 mL) followed by a dropwise addition of 20% aq. NaOH (7.50 ml) at room temperature. The reaction mixture was allowed to stir for 16 h and quenched by the careful addition of conc. HCl (6 N, 1.7 mL). The mixture was extracted with hexanes (2×20 mL) and the combined organic layer was washed with H₂O (3×20 mL). The combined organic layer was dried over sodium sulfate, filtered, and concentrated in vacuo to yield a crude residue. The residue was purified by silica-gel column chromatography (hexanes:EtOAc; 100:0 to 90:10) to yield (+)-8 (2.80 g, 49% yield) as a colorless oil. The ¹H and ¹³C-NMR of the product are consistent with the values reported in literature and commercial (−)-8.

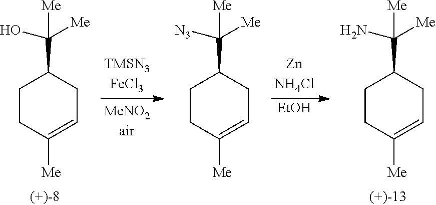 [SMILES: "EtOH" ; CC1=CC[C@H](C(C)(C)O)CC1 ; CC1=CC[C@H](C(C)(C)N=[N+]=[N-])CC1 ; CC1=CC[C@H](C(C)(C)N)CC1]

(+)-13: An identical two-step procedure used to convert (+)-8 to (+)-13 was used to convert (+)-8 (3.00 g, 19.4 mmol) to (+)-13 (900 mg, 30% yield). All spectral data were consistent with literature values and commercial (−)-13.

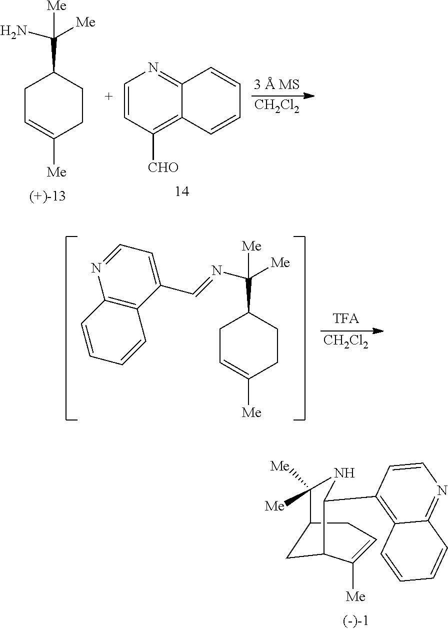

Aristoquinoline, (−)-1: Quinoline-4-carboxaldehyde 14 (79.0 mg, 0.503 mmol) was added in a single portion to a stirred solution of the (−)-13 (73.0 mg, 0.476 mmol) and 3 Å molecular sieves in anhydrous CH₂Cl₂ (4 mL) under an atmosphere of N₂. The reaction was allowed to stir for 16 h and quenched by the addition of aq. NaHCO₃ (10 mL) and extracted with CH₂Cl₂ (3×20 mL). The combined organic phases were dried and concentrated in vacuo to give an oily residue. A solution of the residue in anhydrous CH₂Cl₂ (2 mL) was transferred to a stirred solution of TFA (1 mL, 13.1 mmol) in anhydrous CH₂Cl₂ (3 mL). The reaction was allowed to stir for 30 mins, followed by the careful addition of aq. NaHCO₃ until basic. The aqueous layer was extracted with CH₂Cl₂ (3×20 mL) and the combined organic phases were dried over sodium sulfate, filtered, and concentrated in vacuo to yield an oily residue. The crude was purified by silica gel chromatography (CH₂Cl₂:MeOH:NH₄OH; 89:1:10) to afford the free base of (−)-1 as a colorless oil (68 mg, 49% yield). For X-ray crystallography, the free base of (−)-1 was converted to its dihydrochloride salt by dissolving in Et₂O and treating dropwise with an ethereal HCl solution until turbidity persisted. The Et₂O and excess HCl were removed in vacuo to leave a white solid, which was recrystallized in EtOH/EtOAc.

¹H NMR: (400 MHz, CDCl₃) δ 8.78 (d, J=4.6 Hz, 1H), 8.10 (dd, J=8.4, 1.3 Hz, 1H), 8.07 (dd, J=8.5, 1.3 Hz, 1H), 7.69 (ddd, J=8.4, 6.8, 1.5 Hz, 1H), 7.58 (ddd, J=8.3, 6.8, 1.5 Hz, 1H), 7.19 (d, J=4.6 Hz, 1H), 5.57 (s (broad), 1H), 4.96 (d, J=2.5 Hz, 1H), 2.61 (q, J=3.0 Hz, 1H), 2.47 (dt, J=12.6, 3.4 Hz, 1H), 2.33 (m, 1H), 2.12 (m, 1H), 1.76 (dt, J=12.6, 3.3 Hz, 1H), 1.59 (dt, J=6.5, 3.3 Hz, 1H), 1.38 (s, 3H), 1.27 (s, 3H), 0.66 (dt, J=3.2, 1.7 Hz, 3H). ¹³C NMR: (101 MHz, CDCl₃) δ 150.2, 149.2, 148.2, 132.8, 130.7, 128.9, 126.4, 126.2, 125.0, 123.1, 117.4, 54.3, 53.2, 40.7, 34.6, 30.3, 29.4, 27.8, 25.7, 24.1. HRMS (ESI-TOF) m/z: [M+H]⁺ Calcd for C₂₀H₂₅N₂: 293.2018, found: 293.2013 (1.7 ppm). [α]²⁵_{D}: −189.57 (c 15.2; CHCl₃). Chiral HPLC: 8.136 min (11%), 8.953 min (89%). RP-HPLC: 1.738 min (97%).

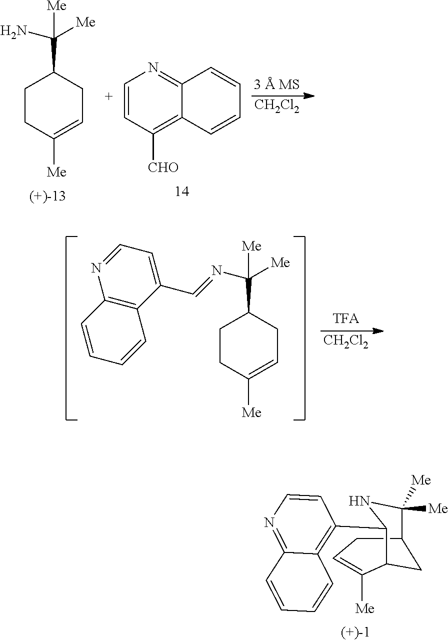

Aristoquinoline, (+)-1: Synthesized using the same procedure as (−)-1 using (+)-13 (100 mg, 0.652 mmol) and 14 (107 mg, 0.681 mmol), to yield (+)-1 as a clear oil (81 mg, 43% yield).

¹H NMR: (400 MHz, CDCl₃) δ 8.79 (d, J=4.6 Hz, 1H), 8.11 (dd, J=8.5, 1.3 Hz, 1H), 8.08 (dd, J=8.5, 1.4 Hz, 1H), 7.69 (ddd, J=8.3, 6.8, 1.4 Hz, 1H), 7.58 (ddd, J=8.3, 6.8, 1.4 Hz, 1H), 7.20 (d, J=4.6 Hz, 1H), 5.57 (dtd, J=3.0, 3.0, 1.6 Hz, 1H), 4.96 (d, J=2.5 Hz, 1H), 2.61 (q, J=2.9 Hz, 1H), 2.47 (dt, J=12.6, 3.1 Hz, 1H), 2.33 (m, 1H), 2.12 (m, 1H), 1.76 (dt, J=12.6, 3.3 Hz, 1H), 1.59 (dt, J=6.4, 3.2 Hz, 1H), 1.38 (s, 3H), 1.27 (s, 3H), 0.66 (dt, J=3.2, 1.7 Hz, 3H). ¹³C NMR: (101 MHz, CDCl₃) δ 150.2, 149.2, 148.2, 132.7, 130.6, 128.9, 126.4, 126.1, 125.0, 123.0, 117.4, 54.3, 53.2, 40.7, 34.6, 30.3, 29.4, 27.7, 25.6, 24.1. HRMS (ESI-TOF) m/z: [M+H]⁺ Calcd for C₂₀H₂₅N₂: 293.2018, found: 293.2013. [α]²⁵_{D}: +202.28 (c 1.23, CHCl₃). Chiral HPLC: 8.241 min (>99%). RP-HPLC: 1.710 min (97%).

### Example 5: Additional Exemplary Syntheses, Yields, and IC₅₀ Values

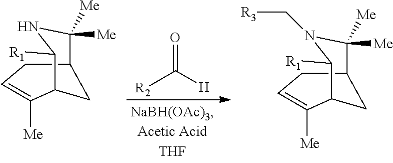

Alkylated Amines: A solution of ARQ-analog (1 equiv) and THF (3 mL) was treated with aldehyde (eq.1.1) and NaCNBH₃ (2 equiv) at room temperature. The reaction was stirred for 10 min. Glacial acetic acid was added and reaction was monitored by TLC until starting material was consumed. Upon completion, the solvent was removed in vacuo, and the resulting solid was diluted with water (5 mL) and basified by addition of sodium bicarbonate solution (25 mL) to a pH of >8. The aqueous layer was washed with 30 mL ethyl acetate. The combined organic layer was washed with saturated NaCl and H₂O and dried over Na₂SO₄, and concentrated under vacuum. The crude product was purified by silica column chromatography using 0-10% MeOH/DCM to afford product.

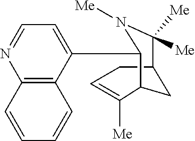

The methylated compound was prepared from aristoquinoline and paraformaldehyde according to the above procedure. Yield 26%, IC₅₀ 1.4 μM.

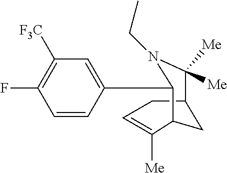

The ethylated compound was prepared from 4-fluoro-3-trifluoromethyl ARQ derivative and acetylaledhyde according to the above procedure. Yield 19%, IC₅₀ 0.76 μM.

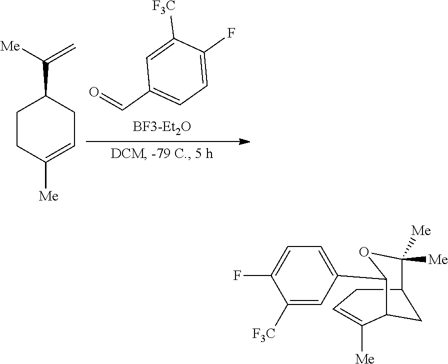

Compounds Containing Oxygen Heterocycles: A solution of (+)-limonene (1 eq). and dichloromethane (4 mL) was treated with BF₃·Et₂O (0.20 eq.) at −79° C. The aldehyde (1.1 eq.) was added dropwise to flask and allowed to react for 2 hours. The reaction was monitored by TLC until starting material was consumed. Upon completion, the solvent was removed in vacuo, and the resulting solid was diluted with EtOAc (30 mL) and was washed by addition of sodium bicarbonate solution (30 mL). The combined organic extract was further washed with saturated NaCl and dried over Na₂SO₄, and concentrated under vacuo. The crude product was purified by silica column chromatography using Hex/EtOAc solvent system to afford the product.

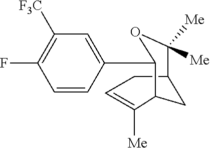

This exemplary compound was prepared according to the procedure above using 4-fluoro-3-trifluoromethylbezaldehyde. Yield 68%, IC₅₀>100 μM.

Additional Exemplary Compounds: Additional exemplary compounds having the following generic structure are shown below.

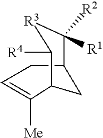

R¹ and R² can independently be hydrogen, methyl, ethyl, isopropyl, cyclopropyl, phenyl, —CD₃, —CF₃, or —CH₂CF₃. R³ is NH, NMe, NEt, NAc, NPh, another alkyl or cycloalkylamine, S, or O, and R⁴ can be selected from:

Exemplary R₄ groups and associated yields and IC₅₀ values are shown in Table 3 below:

TABLE 3
Yields (%) and α3β4 nAChR IC₅₀ Values (μM) for Selected Compounds

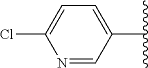 [SMILES: *c1ccc(Cl)nc1]

25%
2.9 μM

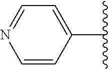 [SMILES: *c1ccncc1]

23%
0.62 μM

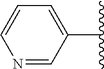 [SMILES: *c1cccnc1]

29%
2.8 μM

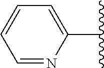 [SMILES: *c1ccccn1]

22%
1.6 μM

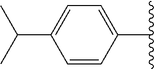 [SMILES: *c1ccc(C(C)C)cc1]

39%
0.39 μM

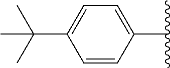 [SMILES: *c1ccc(C(C)(C)C)cc1]

24%
0.66 μM

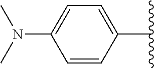 [SMILES: *c1ccc(N(C)C)cc1]

14%
12 μM

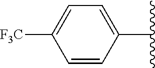 [SMILES: *c1ccc(C(F)(F)F)cc1]

33%
0.79 μM

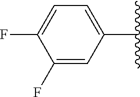 [SMILES: *c1ccc(F)c(F)c1]

35%
0.32 μM

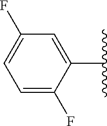 [SMILES: *c1cc(F)ccc1F]

33%
12 μM

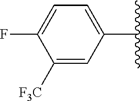 [SMILES: *c1ccc(F)c(C(F)(F)F)c1]

52%
0.28 μM

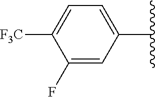 [SMILES: *c1ccc(C(F)(F)F)c(F)c1]

—
—

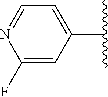 [SMILES: *c1ccnc(F)c1]

49%
>100 μM

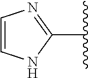 [SMILES: *c1ncc[nH]1]

10%
0.52 μM

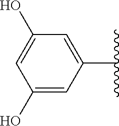 [SMILES: *c1cc(O)cc(O)c1]

10%
>100 μM

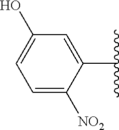 [SMILES: *c1cc(O)ccc1[N+](=O)[O-]]

74%
3.17 μM

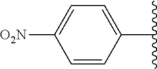 [SMILES: *c1ccc([N+](=O)[O-])cc1]

52%
3.1 μM

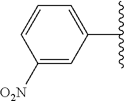 [SMILES: *c1cccc([N+](=O)[O-])c1]

48%
2.0 μM

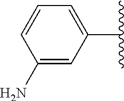 [SMILES: *c1cccc(N)c1]

28%
—

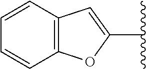 [SMILES: *c1cc2ccccc2o1]

10%
23.29 μM

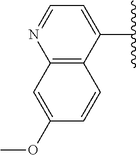 [SMILES: *c1ccnc2cc(OC)ccc12]

21%
52% μM

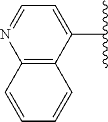 [SMILES: *c1ccnc2ccccc12]

52%^{a}
0.89 μM

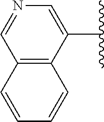 [SMILES: *c1cncc2ccccc12]

8%
0.51 μM

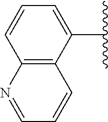 [SMILES: *c1cccc2ncccc12]

7%
0.27 μM

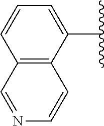 [SMILES: *c1cccc2cnccc12]

4.5%
5.1 μM

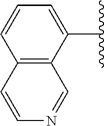 [SMILES: *c1cccc2ccncc12]

20.5%
4.1 μM

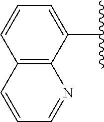 [SMILES: *c1cccc2cccnc12]

29%
0.70 μM
^{a}Aristoquinoline

Further exemplary compounds and α3β4 nAChR IC₅₀ values are found in Table 4:

TABLE 4
α3β4 nAChR IC₅₀ Values (μM) for Selected Compounds^{a, b}

| 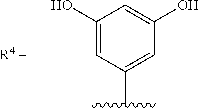 [SMILES: *c1cc(O)cc(O)c1] | 102 | μM |
|---|---|---|
| 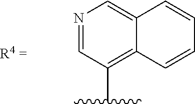 [SMILES: *c1cncc2ccccc12] | 0.81 | μM |
| 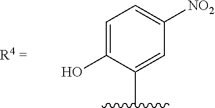 [SMILES: *c1cc([N+](=O)[O-])ccc1O] | 3.17 | μM |
| 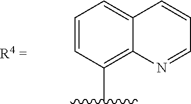 [SMILES: *c1cccc2cccnc12] | | — |
| 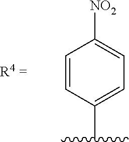 [SMILES: *c1ccc([N+](=O)[O-])cc1] | 3.14 | μM |
| 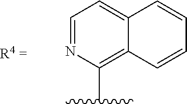 [SMILES: *c1nccc2ccccc12] | 0.70 | μM |
| 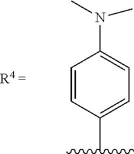 [SMILES: *c1ccc(N(C)C)cc1] | 12.0 | μM |
| 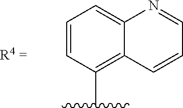 [SMILES: *c1cccc2ncccc12] | 0.27 | μM |
| 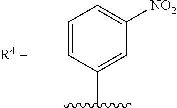 [SMILES: *c1cccc([N+](=O)[O-])c1] | 1.94 | μM |
| 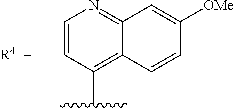 [SMILES: *c1ccnc2cc(OC)ccc12] | 2.17 | μM |
| 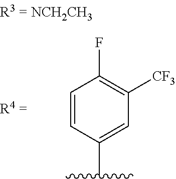 [SMILES: *c1ccc(F)c(C(F)(F)F)c1] | — | |
| 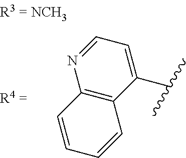 [SMILES: *c1ccnc2ccccc12] | 1.41 | μM |
| 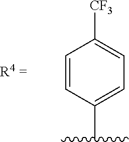 [SMILES: *c1ccc(C(F)(F)F)cc1] | 0.79 | μM |
| 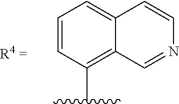 [SMILES: *c1cccc2ccncc12] | 4.10 | μM |
| 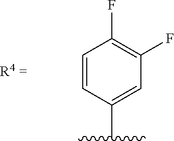 [SMILES: *c1ccc(F)c(F)c1] | 0.32 | μM |
| 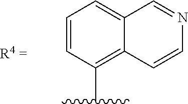 [SMILES: *c1cccc2cnccc12] | 5.10 | μM |
| 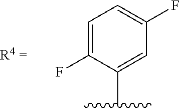 [SMILES: *c1cc(F)ccc1F] | 11.6 | μM |
| 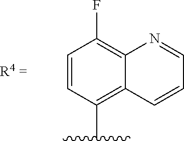 [SMILES: *c1ccc(F)c2ncccc12] | — | |
| 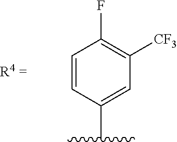 [SMILES: *c1ccc(F)c(C(F)(F)F)c1] | 0.28 | μM |
| 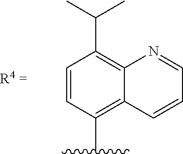 [SMILES: *c1ccc(C(C)C)c2ncccc12] | — | |
| 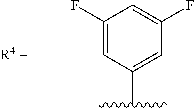 [SMILES: *c1cc(F)cc(F)c1] | — | |
| 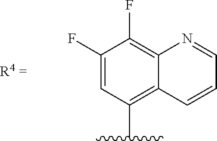 [SMILES: *c1cc(F)c(F)c2ncccc12] | — | |
| 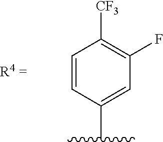 [SMILES: *c1ccc(C(F)(F)F)c(F)c1] | — | |
| 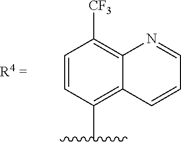 [SMILES: *c1ccc(C(F)(F)F)c2ncccc12] | — | |
| 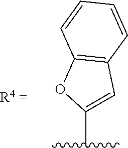 [SMILES: *c1cc2ccccc2o1] | 23.3 | μM |
| 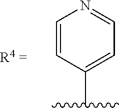 [SMILES: *c1ccncc1] | 0.62 | μM |
| 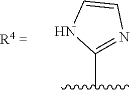 [SMILES: *c1ncc[nH]1] | 0.52 | μM |
| 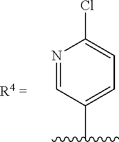 [SMILES: *c1ccc(Cl)nc1] | 2.90 | μM |
| 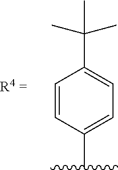 [SMILES: *c1ccc(C(C)(C)C)cc1] | 0.66 | μM |
| 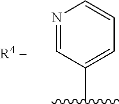 [SMILES: *c1cccnc1] | 2.84 | μM |
| 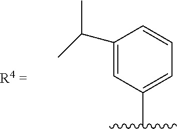 [SMILES: *c1cccc(C(C)C)c1] | 0.39 | μM |
| 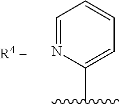 [SMILES: *c1ccccn1] | 1.64 | μM |
| 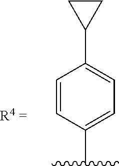 [SMILES: *c1ccc(C2CC2)cc1] | — | |
| 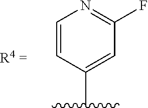 [SMILES: *c1ccnc(F)c1] | >200 | μM |
| 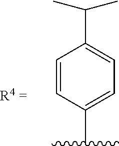 [SMILES: *c1ccc(C(C)C)cc1] | — | |
| 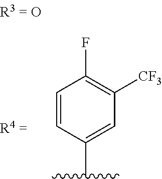 [SMILES: *c1ccc(F)c(C(F)(F)F)c1] | >200 | μM |
| 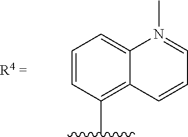 | — | |
| 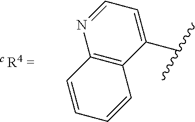 [SMILES: *c1ccnc2ccccc12] | 0.89 | μM |

^{a}R¹ and R² are methyl.
^{b}R³ is NH unless otherwise indicated.
^{c}Aristoquinoline

### Example 6: Deuterium Labeling Experiments

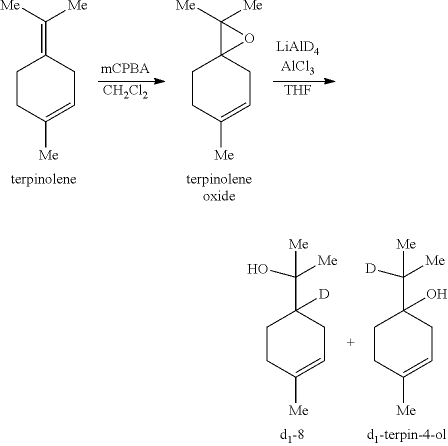

Synthesis of d-8: A solution of m-chloroperbenzoic acid (3.36 g, 70-75%) in anhydrous CH₂Cl₂ (20 mL) was added dropwise to a solution of terpinolene (2.00 g, 14.7 mmol) in anhydrous CH₂Cl₂ (6 mL). After 1 h, the reaction was quenched by the addition of sat. aq. NaHCO₃ (20 mL) and H₂O (20 mL). The organic phase was dried over MgSO₄, filtered and the concentrated in vacuo to yield a residue. The residue was purified by silica-gel chromatography (pentane:Et₂O; 90:10) to yield terpinolene oxide (1.35 g, 61% yield) as a colorless oil. The ¹H- and ¹³C-NMR were consistent with the values reported in literature.

In a modified procedure by Gurudutt et al., lithium aluminum deuteride (65.0 mg, 1.55 mmol) was added to a stirred solution of aluminum chloride (69.0 mg, 0.518 mmol) in anhydrous THF (6 mL) at 0° C. for 15 minutes. To this stirred solution, a solution of terpinolene oxide (790 mg, 5.19 mmol) in anhydrous THF (4 mL) was added dropwise and the reaction was allowed to warm to room temperature and stirred for 16 h. The reaction was quenched by the careful addition of H₂O (3 mL) at 0° C. and the suspension was filtered over Celite and extracted with Et₂O (3×30 mL). The combined organic layer was dried over MgSO₄, filtered, and concentrated in vacuo to yield a residue. The residue was purified by silica-gel chromatography (hexanes:EtOAC; 98:2) to yield dr-8 (65 mg, 8.1% yield) and dr-terpin-4-ol (279 mg, 35% yield).

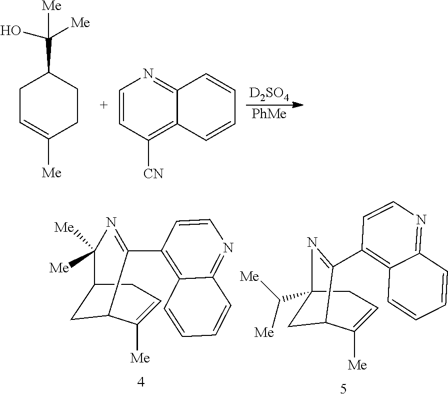

Ritter-Like Reaction using D₂SO₄: Concentrated D₂SO₄ (1.4 mL, 25.59 mmol) was added dropwise to a stirred solution of 4-cyanoquinoline (464 mg, 3.01 mmol) in anhydrous toluene (4 mL) under an atmosphere of N₂ at 0° C., after which a solution of 8 (1.8577 g, 12.04 mmol) in anhydrous toluene (1 mL) was added. The reaction was allowed to warm to room temperature and stirred for 24 h. The reaction was diluted by the careful addition of H₂O (5 mL) and extracted with Et₂O (4×10 mL). The aqueous layer was basified with aqueous NaOH (5 M) to pH>10 and extracted with CH₂Cl₂ (4×10 mL). The CH₂Cl₂ layers were combined, dried over sodium sulfate, filtered, and concentrated in vacuo to give a crude residue, which was purified using silica gel chromatography (hexanes:EtOAc, 100:0 to 0:100) to give 4 (76.6 mg; 8.8% yield) and 5 (33.4 mg; 3.8% yield) as colorless oils.

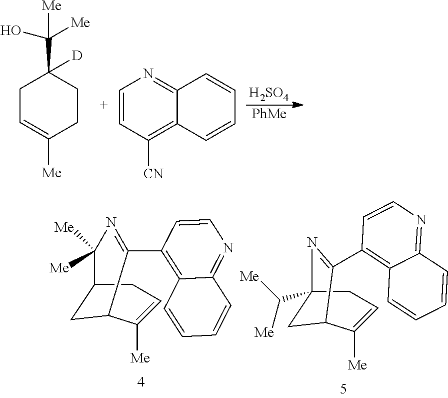

Ritter-Like Reaction on d-8: A solution of d-8 (130 mg, 0.837 mmol) in anhydrous toluene (1 mL) was added dropwise to a stirred solution of sulfuric acid (0.4 mL, 7.14 mmol) and quinoline-4-carbonitrile (129 mg, 0.837 mmol) in anhydrous toluene (5 mL) under an atmosphere of N₂ at 0° C. The reaction was allowed to warm to room temperature and stirred for 24h. The reaction was quenched by the careful addition of H₂O (5 mL) at 0° C. and extracted with Et₂O (2×50 mL). The aqueous layer was basified with aqueous NaOH (10% w/v) to pH=14 and extracted with CH₂Cl₂ (3×30 mL). The organic layers were combined, dried over sodium sulfate, filtered, and concentrated in vacuo to give a crude residue, which was purified using silica gel chromatography (hexanes:EtOAc, 100:0 to 0:100) to give 4 (15 mg; 6.2% yield) and 5 (10 mg; 4.1% yield) as colorless oils.

### Example 7: Calcium Influx Assay

Assay Description. HEK293 cells expressing the rat α3β4 nAChR were loaded with an intracellular, calcium-sensitive dye. Activation of the receptor results in an increase in the intracellular calcium concentration and a corresponding increase in fluorescent signal. Multiple concentrations of test compounds were first applied to the rα3β4-HEK293 cells and monitored for their ability to activate α3β4 nAChRs. Following a 20-minute incubation, 100 nM (±)-epibatidine was then applied to the cells to assess the inhibitory activity of the test compounds. This process allowed us to sequentially measure each compounds' half maximal effective concentration (EC₅₀) and half maximal inhibitory concentration (IC₅₀).

Cell culture. HEK-293 cells expressing rat α3β4 nicotinic acetylcholine receptors were cultured in Dulbecco's modified Eagle's medium, supplemented with 10% fetal bovine serum, G-416 (0.6 mg/mL), penicillin (100U/mL), streptomycin (100 μg/mL), and were maintained in an atmosphere of 5% CO₂ at 37′C. For fluorescence assays, the cells were seeded into 96-well flat bottom, black wall plates, at a density of approximately 50,000 cells per well in 75 μL of media.

Cells seeded at this density grew into a confluent monolayer in 24 h. Ca²⁺ fluorescence assays. Ca²⁺ uptake was determined using a FLIPR calcium 6 assay.

For dye loading, the fluorescent dye was reconstituted in 10 mL assay buffer according to the manufacturer's protocol. 75 μL of the dye solution was added to each well and the plate was incubated at 37° C. for 1 h. Plates were then transferred to a FlexStation 3 maintained at 37° C.

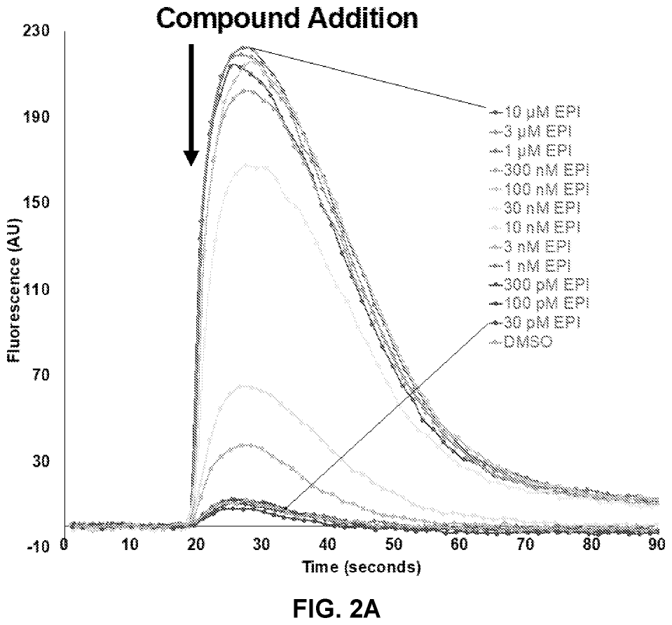

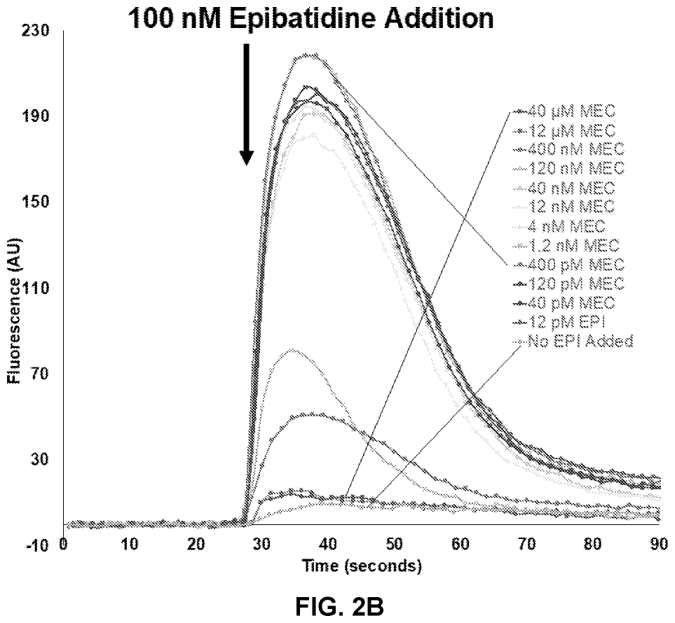

Dual Agonist/AntagonistAssay. For calcium measurements, fluorescence measurements (Ex: 485 nM, Em: 525, Cutoff: 515 nM) were taken for 20 seconds prior to addition of compounds (50 μL of HBSS+0.1% DMSO solution at 5× final concentration) using a FlexStation 3 followed by 90 seconds of continuous measurements. For agonist assays, 12 concentrations of test compounds were added to the cells (300 μM-100 μM, final concentrations). Epibatidine (30 μM -10 μM) and mecamylamine (150 μM-50 μM) were used as agonists and antagonist positive controls (FIGS. 2A-2B).

For subsequent antagonist assays, epibatidine (100 nM, final concentration in HBSS) was added to each well 20 min after the initial agonist assay. Once again, the fluorescence measured for 28 seconds prior to addition and 90 seconds afterwards.

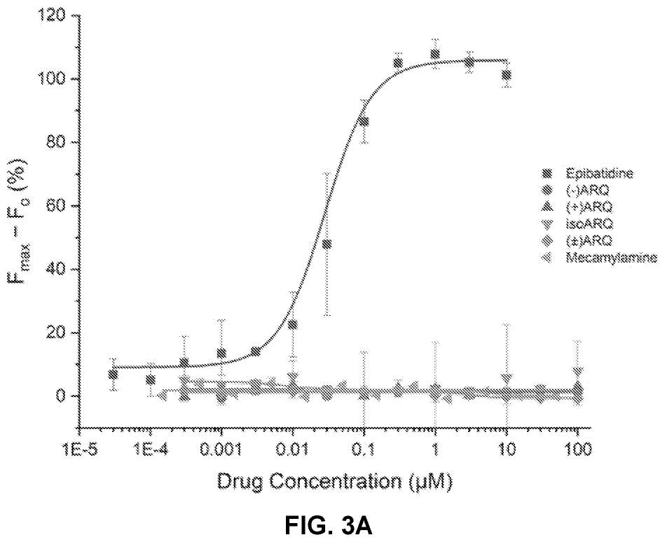

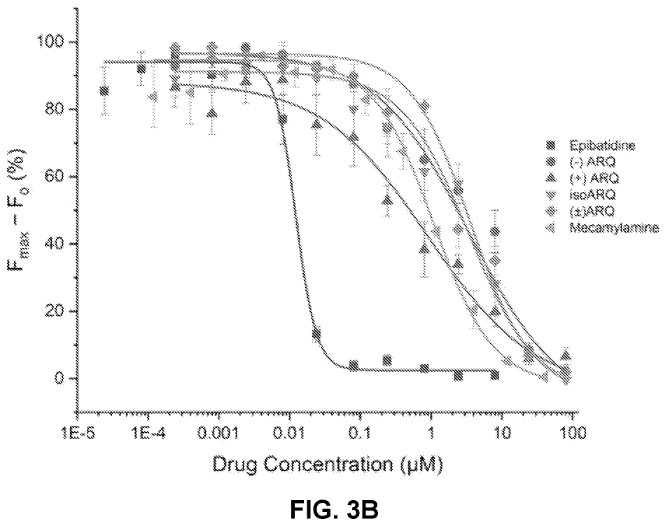

Data reduction. Ligand response for both agonist and antagonist data was calculated by subtracting the average of the pre-addition fluorescence (Fₒ) from the maximum response (Fₘₐₓ) for each well, correcting to a DMSO control, and normalizing to the maximal response produced by epibatidine. Each concentration-response point represents a minimum of two technical replicates and three biological replicates. EC₅₀, Eₘₐₓ, and IC₅₀ values were calculated from corresponding concentration-response curves (see FIGS. 3A-3B) fitted using the logistical curve in OriginPro.

### Example 8: X-Ray Crystallography

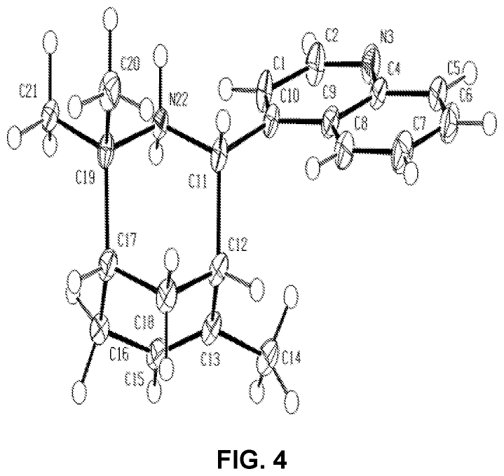

The dihydrochloride salt of (−)-1 was recrystallized in EtOH/EtOAc via slow evaporation. A small crystal measuring roughly 0.1 mm on edge was selected and rotated over 360° to collect intensity data at a wavelength of 0.61991 Å at beamline 21-ID-D, Advanced Photon Source, Argonne National Laboratory on an Eiger 9M detector. The unit cell was determined to be monoclinic, space group P2₁ (No. 4), with unit cell parameters of a=12.803(9), b=18.412(8), c=14.971(7), β=90.99(3)⁰ and four independent molecules in the asymmetric unit. Structure solution and refinement led to R(F)=0.0478. The Flack parameter x=−0.02(2). The structure has been deposited with the CCDC: deposition number 2084820. An exemplary X-ray structure is shown in FIG. 4.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Anderson, B. F.; et al, Aristoteline, a novel indole alkaloid: X-ray structural determination. J. Chem. Soc., Chem. Commun. 1975, 511-512.
2. Arias, H. R.; et al, Alkaloids Purified from Aristotelia chilensis Inhibit the Human alpha3beta4 Nicotinic Acetylcholine Receptor with Higher Potencies Compared with the Human α4β2 and α7 Subtypes. J. Nat. Prod. 2019, 82, 1953-1960.
3. Bick, I. R. C.; et al, Pe-duncularine, an indole-pyrrolizidine alkaloid. J. Chem Soc. D. 1971, 1155-1156.
4. Carman, R. M. et al; Aust. J. Chem. 1994, 47, 195-202.
5. Cespedes, C.; et al, A quinoline alkaloid from Aristotelia chilensis. Phytochemistry 1993, 34, 881-882.
6. Coombs, J. R. et al; J. Org. Chem. 2017, 82, 7040-7044.
7. Creed, S. M.; et al, Isolation and Pharmacological Characterization of Six Opioidergic Picralima nitida Alkaloids. J. Nat. Prod. 2021, 84, 71-80.
8. Darbre, T.; et al, Synthesis of (−)-Hobartine and Related Indole Alkaloids. Helv. Chim.

### Acta 1984, 67, 1040-1052.

9. Dobler, M.; et al, Synthesis of Aristotelia-type alkaloids. Part XI. Total syntheses of (+)-sorelline and (+)-aristolasene. Tetrahedron: Asymmetry 1992, 3, 1411-1420.
10. Dryzhakov, M.; et al, Nitro-Assisted Bronsted Acid Catalysis: Application to a Challenging Catalytic Azidation. J. Am. Chem. Soc. 2015, 137, 9555-8.
11. Gribble, G. W.; et al, Stereocontrolled total synthe-ses of (−)-hobartine and (+)-aristoteline via an intramolecular nitrone-olefin cycloaddition. J. Org. Chem. 1985, 50, 5900-5902.
12. Gurudutt, K. N.; et al, Reduction of Tetrasubsti-tuted Oxiranes. Synthesis 1983, 1983, 888-889.
13. Joshi, B. N.; et al, Terpenoids-LVIII: Base catalysed reactions with N-lithioethylenediamine. Tetrahedron 1964, 20, 2911-2919.
14. Kyburz, R.; et al, Sorelline and Hobartine, Two New Indole alkaloids from Aristotelia peduncu-laris. Helv. Chim. Acta 1979, 62, 2539-2546.
15. Miettinen, K.; et al, The seco-iridoid pathway from Catharanthus roseus. Nat. Commun. 2014, 5, 3606.
16. Mirand, C.; et al, A synthetic entry in the Aristotelia alkaloids. J. Org. Chem. 1982, 47, 4169-4170.
17. Ralph, I.; et al, Chapter 3 Aristotelia Alkaloids. In The Alkaloids: Chemistry and Pharmacology, Vol. 24; Brossi, A., Ed.; Academic Press, 1985; pp 113-151.
18. Ros, H.-P.; et al, The Structure of the Alkaloid Peduncularine. Communication no. 172 on organic natural products. Helv. Chim. Acta 1979, 62, 481-487.
19. Samaniego, W. N.; et al, Ritter reaction on terpenoids. Ill. Stereospecific preparation of bicyclic [3.3.1] substi-tuted piperidines. Tetrahedron Lett. 1994, 35, 6967-6970.
20. Stevens, R. V.; et al, Studies on the stereochemistry of nucleophilic additions to tetrahydropyridinium salts. Expeditious stereospecific total syntheses of (+)-makomakine, (+)-aristoteline, and (±)-hobartine. J. Chem. Soc., Chem. Commun. 1983, 384-386.
21. Ung, A. T.; et al, Formation of 3-azabicyclo[3.3.1]non-3-enes: imino amides vs. imino alkenes. Monatsch Chem. 2014, 145, 983-992.
22. Williams, S. G.; et al, An alkaloid-like 3-azabicyclo[3.3.1]non-3-ene library obtained from the bridged Ritter reaction. Tetrahedron 2017, 73, 116-128.
23. Yuasa, Y.; et al, A Practical Synthesis of d-α-Terpineol via Markovnikov Addition of d-Limonene Using Trifluoroacetic Acid. Org. Process Res. Dev. 2006, 10, 1231-1232.

1. A compound of Formula I or a pharmaceutically acceptable salt thereof: 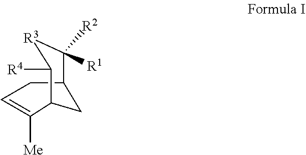
wherein R¹ and R² are independently selected from hydrogen, C1-C4 alkyl, C1-C4 haloalkyl, C3-C6 cycloalkyl, phenyl, and deuterated C1-C4 alkyl, or wherein R¹ and R² together are part of a cycloalkyl group;
wherein R³ is selected from NR⁵, S, and O;
wherein R⁵ is selected from hydrogen, C1-C4 alkyl, and acetyl; and
wherein R⁴ is selected from (1) a C1-C4 alkyl or alkenyl group substituted with a substituted or unsubstituted aryl group or (2) a substituted or unsubstituted 5 to 10-membered aryl or heteroaryl group;
provided that when R¹ and R² are methyl and R³ is NH, R⁴ is not

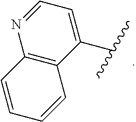 [SMILES: *c1ccnc2ccccc12]

2. The compound of claim 1, wherein R¹ and R² are independently selected from hydrogen, methyl, and ethyl.

3. The compound of claim 1, wherein R¹ and R² are methyl.

4. The compound of claim 1, wherein R¹ and R² together are part of a cyclopropyl group.

5. The compound of claim 1, wherein R³ is NR⁵ and R⁵ is hydrogen, methyl, or ethyl.

6. The compound of claim 1, wherein R³ is O.

7. The compound of claim 1, wherein R¹ and R² are methyl and R³ is NH.

8. The compound of claim 1, wherein R⁴ is a phenyl group substituted with one or more of: hydroxyl, nitro, amino, halo, C1-C4 haloalkyl, C₁-C₄ alkoxy, or C₁-C₄ alkyl, C3-C6 cycloalkyl, or any combination thereof.

9. The compound of claim 1, wherein R⁴ is a quinoline group substituted with one or more of: hydroxyl, nitro, amino, halo, C₁-C₄ haloalkyl, C₁-C₄ alkoxy, or C₁-C₄ alkyl, C3-C6 cycloalkyl, or any combination thereof.

10. The compound of claim 1, wherein R⁴ is 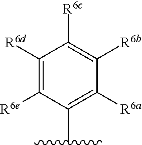
and wherein R^{6a}, R^{6b}, R^{6c}, R^{6d}, and R^{6e} are independently selected from hydrogen, halogen, hydroxyl, N-acetyl, —OCF₃, nitro, isopropyl, cyclopropyl, tert-butyl, dimethylamino, or any combination thereof.

11. The compound of claim 1, wherein R⁴ is 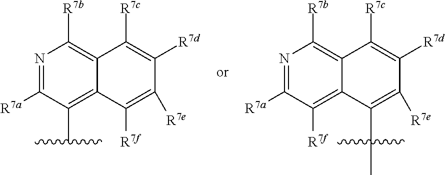
and wherein R^{7a}, R^{7b}, R^{7c}, R^{7d}, R^{7e}, and R^{7f} are independently selected from hydrogen, halogen, hydroxyl, N-acetyl, —OCF₃, nitro, isopropyl, cyclopropyl, tert-butyl, dimethylamino, or any combination thereof.

12. The compound of claim 1, wherein R⁴ is

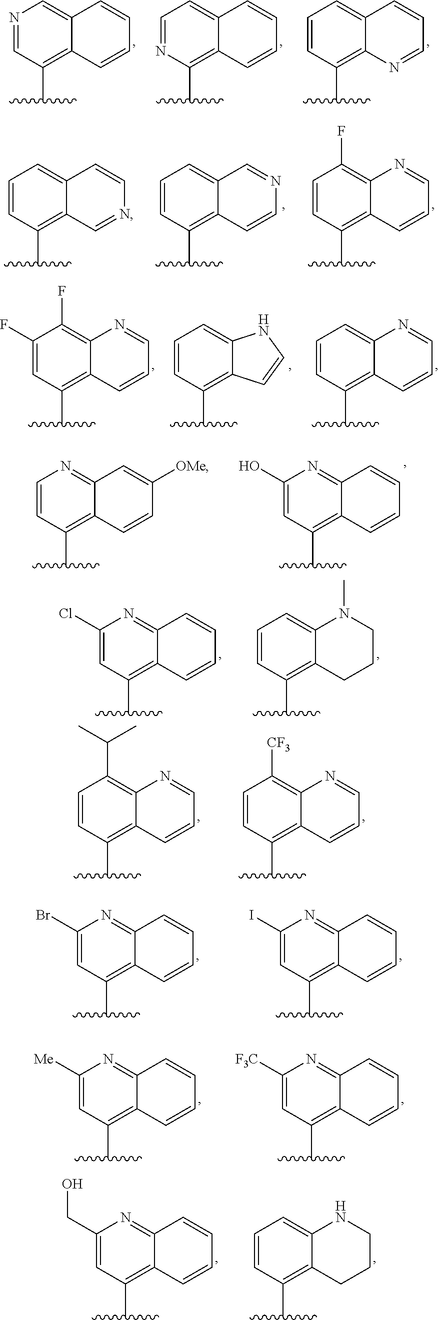

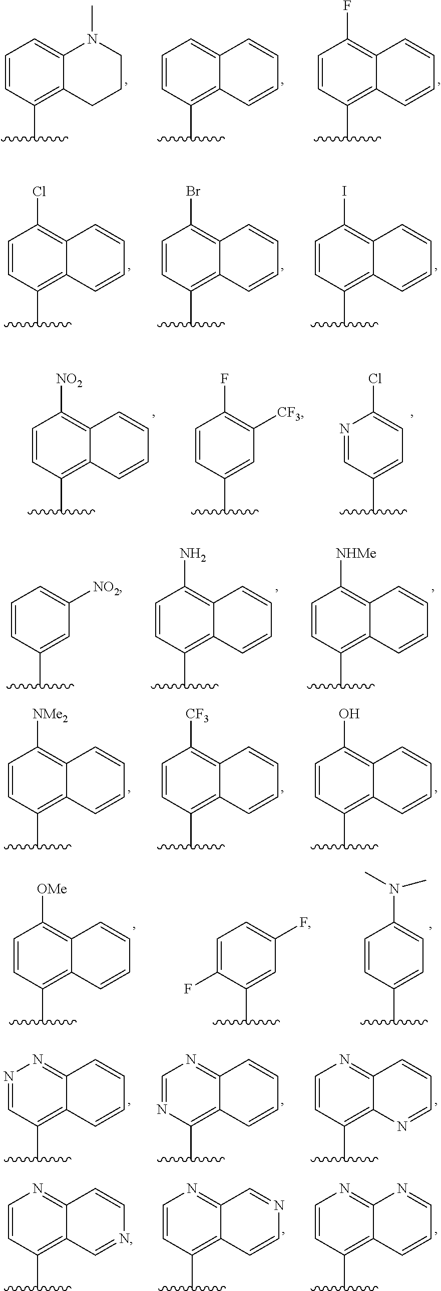

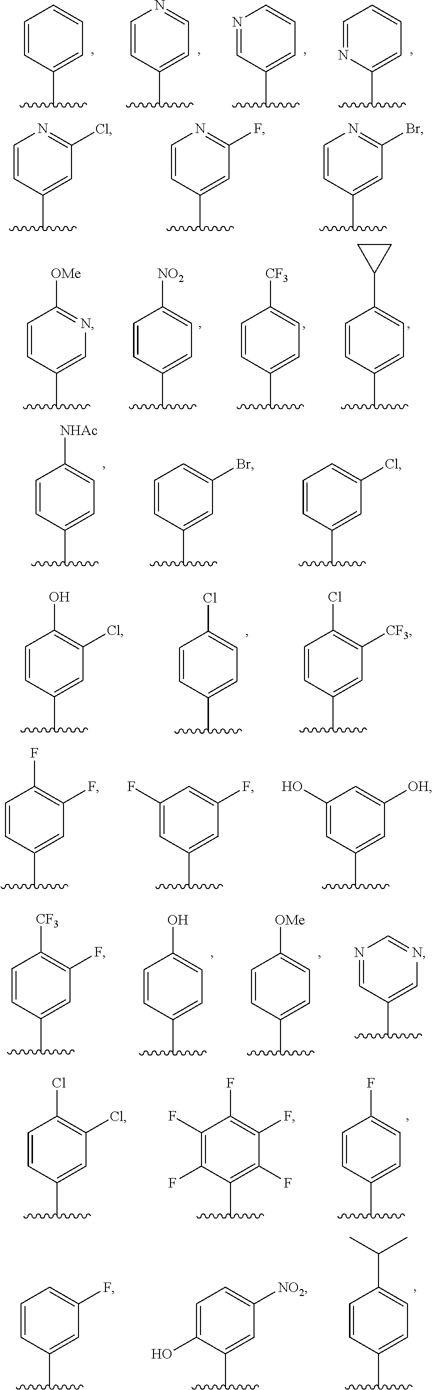

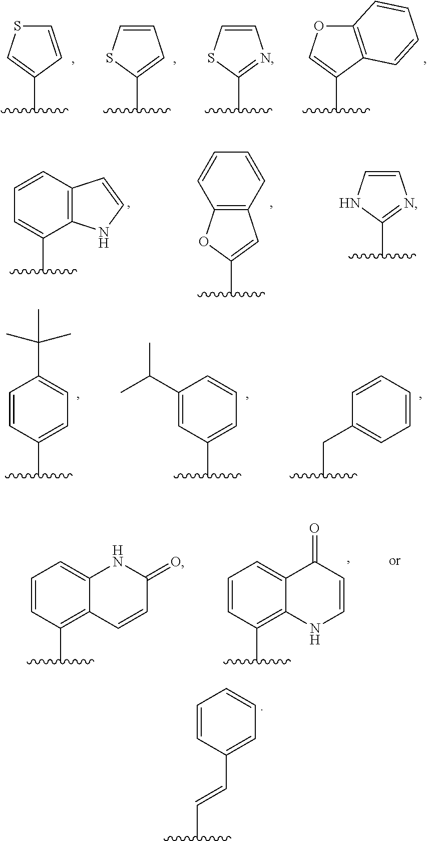

13. The compound of claim 1, wherein the compound of Formula I is selected from 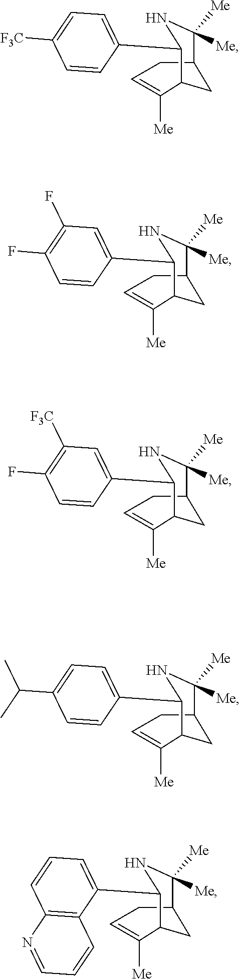
or a pharmaceutically acceptable salt thereof.

14. The compound of claim 1, wherein the compound of Formula I is selected from 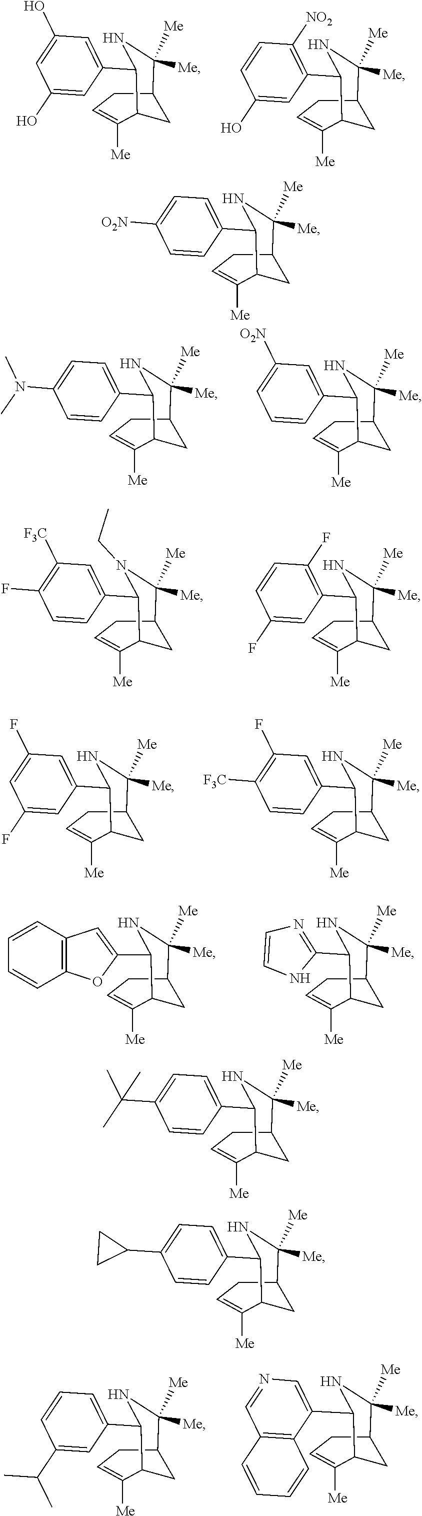 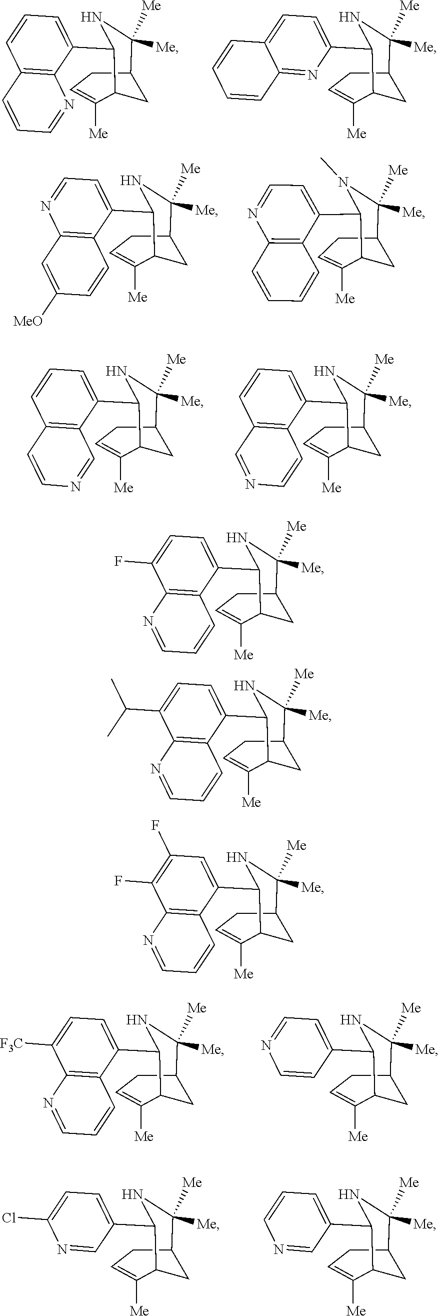 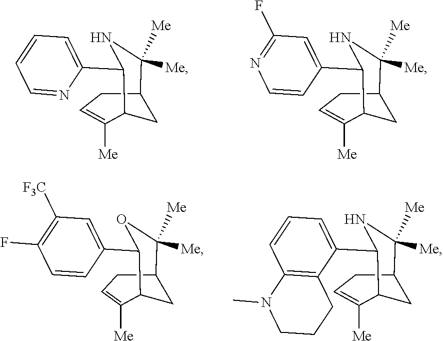
or a pharmaceutically acceptable salt thereof.

15. The compound of claim 1, wherein the compound of Formula I is selected from 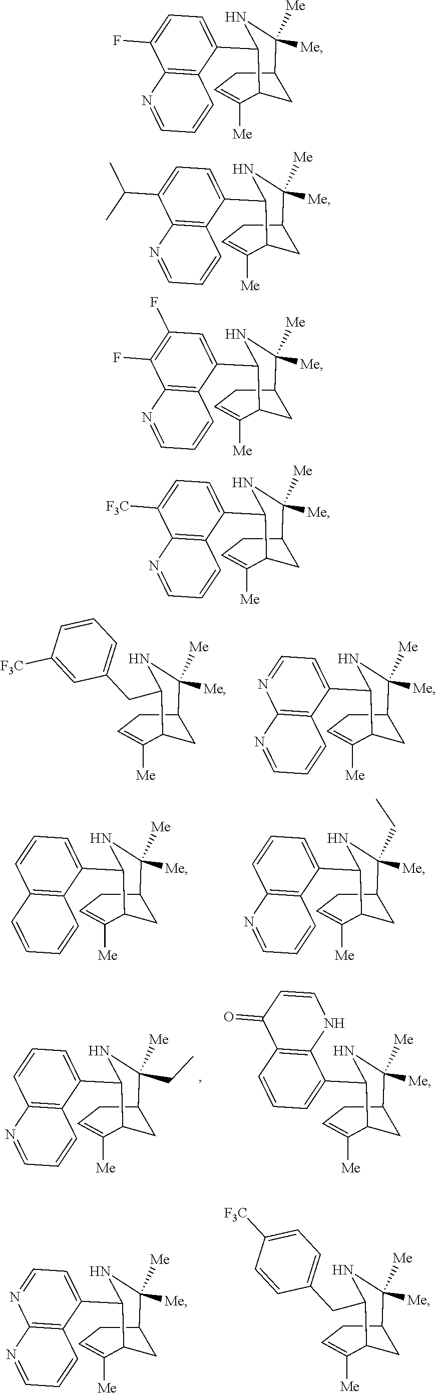 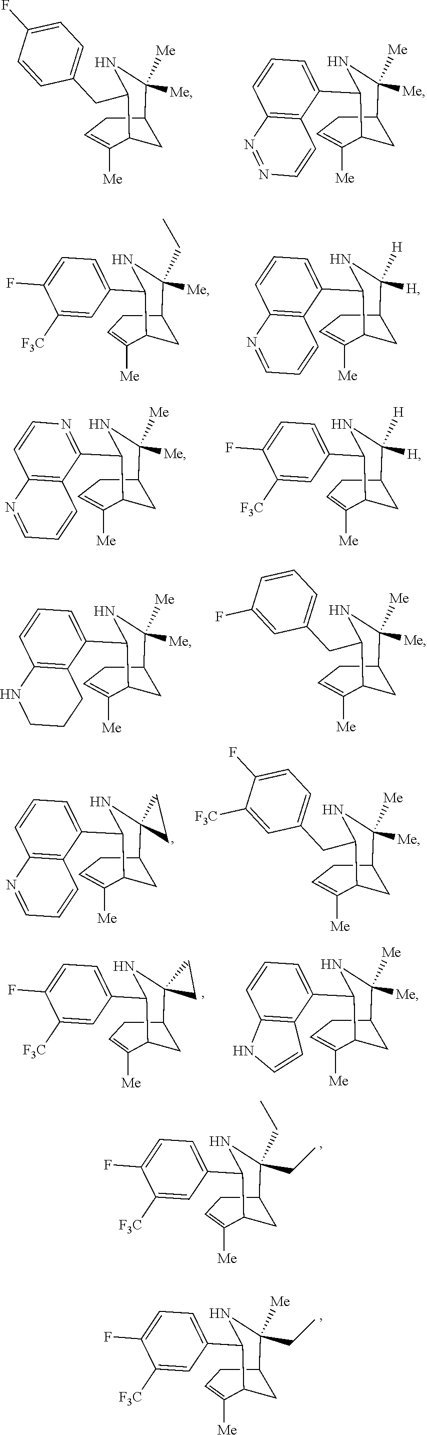 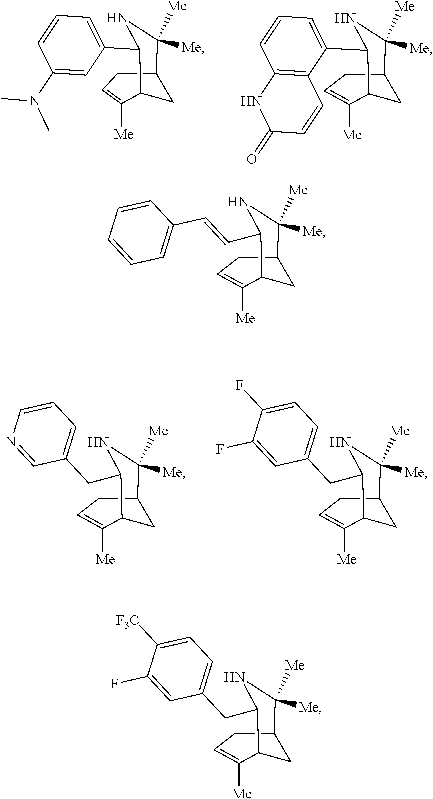
or a pharmaceutically acceptable salt thereof.

16. The compound of claim 1, wherein the compound is a (+) enantiomer, a (−) enantiomer, or any combination thereof.

17. The compound of claim 1, wherein the compound of Formula I has a chiral center a 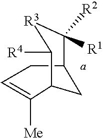
wherein the stereochemistry at chiral center a is substantially R, substantially S, or racemic.

18. A method for inhibiting the activity of at least one nicotinic acetylcholine receptor (nAChR) subtype, the method comprising contacting the nAChR subtype with the compound of claim 1.

19. The method of claim 18, wherein the at least one nAChR subtype is an α3β4 nAChR.

20. The method of claim 18, wherein the compound has an IC₅₀ for the nAChR subtype of less than about 1 μM.

21. The method of claim 18, wherein the compound has an IC₅₀ for the nAChR subtype of from about 0.25 to about 0.5 μM.

22. A pharmaceutical composition comprising the compound of claim 1, an enantiomer thereof, or a pharmaceutically acceptable salt thereof, and a pharmaceutically acceptable carrier or excipient.

23. A method for treating or preventing a disease or disorder associated with aberrant activity of at least one nicotinic acetylcholine receptor (nAChR) subtype in a subject, the method comprising administering a therapeutically effective amount of the compound of claim 1 or the composition of claim 22 to the subject.

24. The method of claim 23, wherein the at least one nAChR subtype is an α3β4 nAChR.

25. The method of claim 23, wherein the subject is a mammal.

26. The method of claim 25 wherein the mammal is a human, dog, cat, rat, mouse, guinea pig, non-human primate, rabbit, or horse.

27. The method of claim 23, where the disease or disorder comprises substance abuse, addiction, or any combination thereof.
